(19) United States
(12) Patent Application Publication
(10) Pub. No.: US 2025/0116666 A1
(43) Pub. Date: Apr. 10, 2025

(54) METHODS FOR USING EXOSOMES TO MONITOR TRANSPLANTED ORGAN STATUS

(71) Applicant: THE TRUSTEES OF THE UNIVERSITY OF PENNSYLVANIA, Philadelphia, PA (US)

(72) Inventor: Prashanth Vallabhajosyula, Bryn Mawr, PA (US)

(73) Assignee: THE TRUSTEES OF THE UNIVERSITY OF PENNSYLVANIA, Philadelphia, PA (US)

(21) Appl. No.: 18/985,464

(22) Filed: Dec. 18, 2024

Related U.S. Application Data
(63) Continuation of application No. 15/934,368, filed on Mar. 23, 2018.
Application No. 15/934,368 is a continuation-in-part of application No. 15/325,466, filed on Jan. 11, 2017.
(60) Provisional application No. 62/025,858, filed on Jul. 17, 2014.

(51) Int. Cl. G01N 33/68

(52) U.S. Cl. CPC G01N 33/56977; G01N 33/56966; G01N 33/6872; G01N 33/6887; G01N 33/6893; G01N 2333/4703; G01N 2333/4712; G01N 2333/70539; G01N 2800/245

(57) ABSTRACT

This present disclosure relates to the use of donor organ-derived microvesicles to monitor the status of a transplanted organ in a subject. Accordingly, this disclosure provides for methods and kits for isolating, purifying and/or identifying donor organ-derived microvesicles from a biological sample of a subject. In certain embodiments, a method for isolating, purifying and/or identifying donor organ-derived microvesicles includes obtaining a biological sample from the subject and isolating, purifying or identifying a donor organ-derived microvesicle from the biological sample by the detection of a protein specific for the donor.

### CROSS-REFERENCE TO RELATED APPLICATIONS

This application is a continuation of U.S. patent application Ser. No. 15/934,368, filed on Jan. 11, 2017, which is a continuation-in-part of U.S. patent application Ser. No. 15/325,466, filed on Jan. 11, 2017, which is a U.S. National Stage Patent Application under 35 U.S.C. § 371 of International Application No. PCT/US2015/040956, filed on Jul. 17, 2015, which claims priority to U.S. Provisional Patent Application Ser. No. 62/025,858, filed on Jul. 17, 2014, the contents of each of which are incorporated by reference herein in their entirety.

### BACKGROUND

Transplantation of a graft organ or tissue from a donor to a host patient is a feature of certain medical procedures and treatment protocols and can significantly improve patient survival and quality of life. Despite efforts to avoid graft rejection through host-donor tissue type matching, in transplantation procedures, where a donor organ is introduced into a host, immunosuppressive therapy is generally required to the maintain viability of the donor organ in the recipient. However, despite the wide use of immunosuppressive therapy, many patients suffer from complications associated with the transplanted organ, such as acute and chronic rejection, over-immunosuppression and infection.

Early detection of organ rejection is a clinical concern in the care of transplant recipients. Detection of rejection before the onset of graft dysfunction allows successful treatment of this condition with aggressive immunosuppression. In addition, it is equally important to reduce immunosuppression in patients who do not have organ rejection to minimize drug toxicity. Many kidney transplant rejection episodes are detected by periodically measuring the function of the transplanted kidney, for example by using biochemical tests such as assays that measure serum creatinine concentrations. Additionally, in liver transplantation, monitoring of liver enzymes in the serum of the recipient is used to monitor the status of the transplanted liver.

Small microvesicles released by cells are known as exosomes (Thery et al., 2002). Exosomes are tissue and major histocompatibility complex (MHC) specific microvesicles (30-200 nm) with stable RNA cargo reflecting the conditional state of the tissue releasing them. Exosomes also take part in the communication between cells by functioning as transport vehicles for proteins, RNAs, DNAs, viruses and prions. Since their discovery, a growing number of therapeutic applications are in development using exosomes derived from various producing cells, such as dendritic cells (DC), T lymphocytes, tumor cells and cell lines (Thery et al., 2002 and Delcayre et al., 2005). For instance, DC-derived exosomes (also designated dexosomes) pulsed with peptides derived from tumor antigens elicit anti-tumor responses in an animal model for the matching tumor (Zitvogel et al., 1998). Two Phase-I clinical trials using autologous dexosomes for the treatment of lung cancer (Morse et al., 2005) and melanoma (Escudier et al., 2005), respectively, have recently been completed. There exists a critical need for a method for monitoring transplanted organ status.

### SUMMARY

The present disclosure provides methods for monitoring the status of a transplanted organ (e.g., heart, lung, kidney, and islet) in a subject. The present disclosure further provides methods for isolating, purifying and/or detecting donor-derived microvesicles from a biological fluid of a transplant recipient.

In certain embodiments, a method for monitoring the status of a transplanted organ in a subject can include (a) obtaining a biological sample from the subject; and (b) isolating, purifying and/or identifying one or more donor organ-derived microvesicles from the biological sample. In certain embodiments, the method can further include the isolation of tissue-specific microvesicles.

In certain embodiments, a method for the isolation, identification and/or purification of donor organ-derived microvesicles can include (a) obtaining a biological sample from the subject; and (b) isolating, purifying and/or identifying one or more donor organ-derived microvesicles from the biological sample by the detection of a marker specific for the donor. In certain embodiments, the marker can be a protein present on the surface of the microvesicles. In certain embodiments, the protein can be a member of the major histocompatibility complex (MHC) that is specifically present on the surface of the donor organ-derived microvesicles.

In certain embodiments, a method for the isolation, identification and/or purification of donor organ-derived microvesicles can include (a) obtaining a biological sample from the subject; and (b) isolating, purifying and/or identifying one or more donor organ-derived microvesicles from the biological sample by the detection of a marker specific for the organ that was transplanted in the recipient. For example, and not by way of limitation, the protein can be a cell surface protein that is specific to the cell type of the donor organ.

In certain non-limiting embodiments, the biological sample can be a bodily fluid such as blood, urine, saliva, nasotracheal secretions, amniotic fluid, breast milk or ascites. In certain embodiments, the biological sample can be a blood sample. In specific non-limiting embodiments, the microvesicles can be purified, isolated and/or obtained in one or more biological samples from a subject. The use of a bodily fluid as a biological sample makes it possible to eliminate invasiveness of the diagnostic or prognostic procedure, and dramatically reduces the burden of the examination on the subject.

The disclosure also provides kits for monitoring the status of a transplanted organ of a subject, where the kit containing reagents useful for isolating, purifying and/or identifying the donor organ-derived microvesicles in a biological sample.

In certain embodiments, the transplanted organ is a heart. In certain embodiments, the transplanted organ is a lung. In certain embodiments, the transplanted organ is a kidney. In certain embodiments, the transplanted organ is an islet.

### BRIEF DESCRIPTION OF THE DRAWINGS

FIGS. 1A-1C. Xenoislet transplantation. (A) Nanosight in light scatter and fluorescent mode for xenotransplant. (B) Nanosight for negative control. (C) Western blot of microvesicles with anti-human CD63 from 3 xenotransplants (lanes 1-3) and negative control (lane 4).

FIG. 2. Nanosight analysis on fluorescent mode for BALB/c (donor) and B6 (recipient) specific antibody quantum dot analysis is shown for full mismatch and negative control.

FIGS. 3A-3E. Detection and purification of exosomes from human renal transplant recipients. (A) Relevant donor-recipient human leukocyte antigen (HLA) profiles are shown. (B) On Nanosight, HLA-A2 and HLA-B27 positive exosomes were seen in the donor plasma (i), and recipient plasma post-transplant day 4 (iii), but not in the recipient plasma pre-transplant (ii). (C) On Western blot, HLA-A2 bound plasma exosomes fractions from recipient pre-transplant and post-kidney implantation but pre-perfusion of the donor organ was negative for aquaporin 2 (renal epithelial marker), but the recipient post-transplant day 4 sample was positive for aquaporin 2. (D) Urinary exosomes were analyzed for HLA-A2 presence. (E) HLA-A2 bound urinary exosomes fractions from postoperative day 4 and day 30 showed expression of renal glomerular marker, podocalyxin-1, but such expression was not observed in the pre-transplant sample.

FIGS. 4A-4D. Detection of transplant human islet signal in recipient mouse plasma exosomes in a xenoislet transplant model. (A) Exosomes were analyzed on the NanoSight NS300 fluorescence mode using anti-HLA-A quantum dot. HLA-A positive signal was detected in xenoislet exosomes (i), human plasma exosomes (positive control) (ii), but not in naive mouse (iii) plasma exosomes (p<0.0001). Exosomes isolated from in vitro human islet culture supernatant also stained positive for HLA-A signal (iv). (B) Similar results were obtained when exosomes were labeled with another human specific MHC, HLA-C quantum dot. (C) Western blot analysis confirmed HLA-A and HLA-C expression in total plasma exosome pool of xenoislet mouse sample, in vitro islet culture supernatant exosomes, human plasma exosomes, but not in naive mouse plasma exosomes. (D) Islet graftectomy was performed in 6 xenoislet animals. In plasma exosome sample from day 21 after islet graftectomy, the HLA-A quantum dot signal was undetectable on NanoSight.

FIG. 5. Heterotopic heart transplantation model. Full MHC-1 mismatched BALB/c hearts (MHC-1 H2-kd) were heterotopically transplanted into strain controlled C57BL/6 (MHC-1 H2-kb) mice (rejection arm, n=64) or immunodeficient C57BL/6 PrkdcSCID (MHC H2-Kb) mice (maintenance arm, n=28). Animals of both study arms were monitored for allograft viability and were sacrificed at various time points for plasma exosome analysis (days 1 to 30).

FIGS. 6A-6B. Donor BALB/c MHC is specifically expressed on BALB/c tissues and their exosomes. (A) Western blot analysis of plasma exosomes from BALB/c and C57BL/6 samples is shown. H2-Kd specificity was only seen in BALB/c samples, not in C57BL/6 samples. Samples tested on Western blot showed exosomes markers CD63 and flotillin-1, but absence of cellular/apoptotic body marker cytochrome c. (B) Nanoparticle detector analysis for anti-BALB/c (H2-Kd) antibody specificity is shown. A distinct H2-Kd signal was seen in BALB/c donor plasma samples, not in C57BL/6 samples or IgG isotype controls.

FIG. 7. Allograft viability by Kaplan Meier. Animals were followed for cardiac allograft viability 1-30 days.

FIGS. 8A-8B. Recipient plasma exosome profiles during acute rejection on the NanoSight Nanoparticle detector. Total recipient plasma exosome quantities (blue) versus recipient donor MHC-1-specific pool (red) normalized to exosome protein quantity is shown for (A) maintenance arm and (B) rejection arm.

FIGS. 9A-9B. Acute rejection leads to distinct changes in donor pool but not total exosomes quantity. Each data point represents a single animal. (A) There were no differences in total plasma exosome quantity (p=0.278 for rejection arm, p=0.157 for maintenance arm). (B) On day 1, donor heart exosome signals were similar between the two groups (p=0.280), however significant decrease in the signal was noted by 2 in the Rejection arm (p=2x10-4). Donor exosome signal further decreased on day 3 (p=3x10-6 compared to day 1; p=6x10-5 compared to day 2) but was unchanged in the Maintenance arm. With acute rejection, donor exosome signal remained low and unchanged after day 3 (p=0.217 for day 3 to day 30), similar to the pretransplant levels.

FIGS. 10A-10B. Histologic ISHLT grading of acute allograft rejection. Corresponding H&E sections are shown with CD3 stains for (A) maintenance versus (B) rejection arm.

FIG. 11. Characterization of test accuracy. Receiver operating characteristic (ROC) curves for donor heart-specific exosome signal (AUC-0.934+0.030), total exosome quantity (AUC-0.659+0.080), median exosome size (AUC=0.677+0.085), and mean exosome size (AUC-0.679+0.083). Donor heart exosome profiling showed that a signal threshold cut-off of <0.3146% was 91.4% sensitive and 95.8% specific for diagnosing acute rejection.

FIG. 12. Transplant heart specific exosomes contain cardiac myocyte specific marker protein Troponin I. THEs were purified from plasma exosome pool using anti-donor HLA antibody conjugated beads and the protein content was analyzed by Western blot for expression of Troponin I. Representative samples from 2 patients are shown above. In Patient 2, AMR was noted on day 14 heart biopsy, but no AMR was seen in the other patients. THEs from day 7 showed expression of complement C4 only in Patient 2, with lower expression on day 14 sample. C4 was not seen in THE samples from the other 4 patients. IgG isotype antibody bead bound exosomes and anti-donor antibody bead bound exosomes from pretransplant samples are shown as negative controls.

FIGS. 13A-13B. Donor lung specific exosome profiles enable noninvasive diagnosis of acute rejection of lung allograft. (A) Transplant lung specific exosome signal (mean+standard deviation) in Lewis recipient rats undergoing orthotopic left lung transplantation across full major histocompatibility mismatch is shown (n=12). Donor exosome signal was significantly decreased and reached baseline signals by post-transplant day 3 (p<0.001). (B) Wide field fluorescence microscopy of allograft for GFP signals is shown for post-transplant time points 4 hours, days 1 and 3. Donor was a transgenic rat expressing human CD63-GFP on its exosomes. Day 3 graft-draining lymphoid tissue also showed positivity for donor lung exosomes.

### DETAILED DESCRIPTION

The present disclosure provides techniques related to the use of microvesicles, identified herein, to monitor the status of a transplanted organ in a subject. The present disclosure, in certain embodiments, further provides methods for isolating, purifying and/or identifying donor organ-derived microvesicles released into the bodily fluids of a subject. In certain embodiments, the present disclosure provides for methods and kits for isolating, purifying and/or identifying one or more donor organ-derived microvesicles in a biological sample of a subject.

Medical practices rely on histological or clinical parameters to monitor the status and/or function of transplanted hearts and lungs, and there are no existing methods of non-invasively detecting monitoring transplanted hearts or lungs. For example, for patients undergoing cardiac transplantation, surveillance endomyocardial biopsies are taken at weekly intervals to 6 weeks, then at 2 weekly intervals until 3 months. In addition, any positive biopsy is followed-up by a repeat biopsy one week later to ensure that anti-rejection therapy has been successful. Patients also undergo further biopsies when clinically indicated, resulting in a patient undergoing multiple biopsies within the first year of transplantation. Therefore, detection of donor organ-derived microvesicles circulating in a biological fluid of a recipient, as described herein, provides for a non-invasive and time-sensitive assay to monitor the status of a transplanted organ, especially, in the case of, transplanted heart and lungs.

### Definitions

As used herein, “transplantation” refers to the process of taking a cell, tissue or organ, called a “transplant” or “graft” from one subject and placing it or them into a (usually) different subject. The subject who provides the transplant is called the “donor” and the subject who received the transplant is called the “recipient.” An organ, or graft, transplanted between two genetically different subjects of the same species is called an “allograft.” A graft transplanted between subjects of different species is called a “xenograft.” Examples of transplanted organs that can be monitored by the methods disclosed herein include, but are not limited to, heart, lungs, kidney, liver, islets and pancreas.

As used herein, the term “biological sample” refers to a sample of biological material obtained from a subject, e.g., a human subject, including a biological fluid, e.g., blood, plasma, serum, urine, sputum, spinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid, bronchoalveolar fluid, biliary fluid and combinations thereof. In certain non-limiting embodiments, the donor organ-derived microvesicles are isolated and/or purified from a blood sample obtained from a subject. In certain non-limiting embodiments, the donor organ-derived microvesicles are isolated and/or from a urine sample obtained from a subject.

The term “patient” or “subject,” as used interchangeably herein, refers to any warm-blooded animal, e.g., a human. Non-limiting examples of non-human subjects include non-human primates, dogs, cats, mice, rats, guinea pigs, rabbits, fowl, pigs, horses, cows, goats, sheep, etc.

The term “microvesicle” as used herein, refers to vesicles that are released from a cell. In certain embodiments, the microvesicle is a vesicle that is released from a cell by exocytosis of intracellular multivesicular bodies. In certain embodiments, the microvesicles can be exosomes.

As used herein, the term “transplant rejection,” is defined as functional and/or structural deterioration of an organ. Transplant rejection can include functional and/or structural deterioration due to an active immune response expressed by the recipient, and independent of non-immunologic causes of organ dysfunction. Transplant rejection can include donor organ injury, such as an infection of the transplant organ.

### Methods

The present disclosure, in certain embodiments, provides methods for monitoring the conditional state of a transplanted organ by monitoring the microvesicles released from the donor organ. In certain embodiments, the present disclosure provides methods for isolating, detecting and/or purifying microvesicles derived from a donor organ transplanted in a recipient for monitoring the conditional state of a transplanted organ. In certain embodiments, the methods of the present disclosure can further include the detection of one or more markers specific to the donor organ to confirm the isolation and/or purification of donor organ-derived microvesicles, e.g., exosomes.

In certain embodiments, the methods for monitoring the conditional state of a transplanted organ in a subject include: (a) obtaining a biological sample from the subject; and (b) isolating, purifying and/or identifying one or more donor organ-derived microvesicles from the biological sample. In certain embodiments, the method can include enriching donor organ-derived microvesicles for microvesicles originating from a specific cell type and/or tissue. In certain embodiments, the method can include confirming that the donor organ-derived microvesicles originated from a specific cell type and/or tissue.

In certain embodiments, a method for the isolation, identification and/or purification of donor-derived microvesicles can include: (a) obtaining a biological sample from the subject; and (b) isolating, purifying and/or identifying one or more donor organ-derived microvesicles from the biological sample by the detection of a marker specific for the donor. In certain embodiments, the marker can be a protein. For example, and not by way of limitation, the protein can be a member of the major histocompatibility complex (MHC) classes of proteins that is specific to the donor, as detailed below.

In certain embodiments, a method for the isolation, identification and/or purification of donor-derived microvesicles can include: (a) obtaining a biological sample from the subject; and (b) isolating, purifying and/or identifying one or more donor organ-derived microvesicles from the biological sample by the detection of a marker specific for the organ that was transplanted in the recipient. For example, and not by way of limitation, the marker can be a microvesicle surface protein that is specific to the cells of the donor organ, e.g., a protein specific to a kidney cell, as detailed below.

In certain embodiments, a method for the isolation, identification and/or purification of donor-derived microvesicles can include (a) obtaining a biological sample from the subject; (b) isolating and/or purifying one or more microvesicles from the biological sample to generate a microvesicle sample; and (c) isolating, purifying and/or identifying one or more donor organ-derived microvesicles from the microvesicle sample by the detection of a marker specific for the organ that was transplanted in the recipient and/or by the detection of a marker specific for the donor. In certain embodiments, the microvesicle sample can include microvesicles derived from the donated organ (or cells thereof) and/or microvesicles derived from a recipient organ (or cells thereof).

In certain embodiments, the donor organ-derived microvesicles can be exosomes. In certain embodiments, the microvesicles can be in the size range from about 30 nm to 1000 nm. For example, and not by way of limitation, the microvesicles can be from about 30 nm to about 900 nm, from about 30 nm to about 800 nm, from about 30 nm to about 700 nm, from about 30 nm to about 600 nm, from about 30 nm to about 500 nm, from about 30 nm to about 400 nm, from about 30 nm to about 300 nm, from about 30 nm to about 200 nm, from about 30 nm to about 100 nm or from about 30 nm to about 50 nm in size.

In certain embodiments, and as noted above, methods for assessing the conditional state of a transplanted organ in the subject and/or the isolation and/or purification of donor-derived microvesicles from a subject include obtaining at least one biological sample from the subject. In certain embodiments, the microvesicles can be detected in blood (including plasma or serum) or in urine, or alternatively at least one microvesicle can detected in one sample, e.g., the blood, plasma or serum, and at least one other microvesicle is detected in another sample, e.g., in urine. The step of collecting a biological sample can be carried out either directly or indirectly by any suitable technique.

For example, and not by way of limitation, a blood sample from a subject can be carried out by phlebotomy or any other suitable technique, with the blood sample processed further to provide a serum sample or other suitable blood fraction.

In certain embodiments, the transplanted organ is a heart. In certain embodiments, the transplanted organ is a lung. In certain embodiments, the methods disclosed herein is used for detecting acute rejection of the transplanted organ (e.g., heart and lung). In certain embodiments, the method disclosed herein has high sensitive and specificity in detecting early acute rejection. In certain embodiments, the sensitivity is about 75-99%. In certain embodiments, the specificity is about 75-99%. In certain embodiment, the sensitivity is about 91%. In certain embodiments, the specificity is about 95%. In certain embodiments, intraexosomal cargo of the isolated donor organ-derived microvesicle is further profiled for expression of cardiac markers, e.g., Troponin ImRNA and protein. The expression of cardiac markers validates donor organ-derived microvesicle enrichment.

In certain embodiments, the information provided by the methods described herein can be used by the physician in determining the most effective course of treatment (e.g., preventative or therapeutic). A course of treatment refers to the measures taken for a patient after the assessment of increased risk for organ rejection is made. For example, when a subject is identified to have an increased risk of organ rejection, the physician can determine whether frequent monitoring of donor organ-derived microvesicles is required as a prophylactic measure.

### Microvesicle Isolation Techniques

Circulating donor-organ derived microvesicles can be isolated from a subject by any means known in the art and currently available. Circulating donor-organ derived microvesicles can be isolated from a biological sample obtained from a subject, such as a blood sample, or other biological fluid. In certain embodiments, the circulating donor-organ derived microvesicles can be isolated from the urine of the recipient. In certain embodiments, the microvesicles can be exosomes.

There are several capture and enrichment platforms that are known in the art and currently available. For example, microvesicles can be isolated by a method of differential centrifugation as described by Raposo et al., 1996. Additional methods include anion exchange and/or gel permeation chromatography as described in U.S. Pat. Nos. 6,899,863 and 6,812,023. Methods of sucrose density gradients or Organelle electrophoresis are described in U.S. Pat. No. 7,198,923. A method of magnetic activated cell sorting (MACS) is described in Taylor and Gercel-Taylor, 2008. A method of nanomembrane ultrafiltration concentrator is described in Cheruvanky et al., 2007. Microvesicles can be identified and isolated from a biological sample of a subject by a newly developed microchip technology that uses a unique microfluidic platform to efficiently and selectively separate microvesicles (Nagrath et al., 2007). This technology can be adapted to identify and separate microvesicles using similar principles of capture and separation.

In certain embodiments, the donor organ-derived microvesicles can be purified, isolated and/or identified by the detection of a marker specific to the donor organ and/or to the cell type of the donated organ. In certain embodiments, the marker can be nucleic acids and/or proteins that reside on the surface or within the microvesicles. The microvesicles, e.g., exosomes, can be isolated from a biological sample and analyzed using any method known in the art. The nucleic acid sequences, fragments thereof, and proteins, and fragments thereof, can be isolated and/or identified in a biological sample using any method known in the art, as described below.

In certain embodiments, the microvesicles isolated from a biological sample can be enriched for those released from the donor organ through the use of the MHC (Major histocompatibility complex) proteins that reside on the surface of the microvesicles. For example, and not by way of limitation, the donor organ-derived microvesicles can be isolated by the detection of MHC proteins specific to the donor microvesicles as compared to the microvesicles released by the recipient, e.g., by the detection of a specific allele of HLA-A and/or HLA-B genes as compared to the recipient. In certain embodiments, the methods of the present disclosure can include identifying the specific MHC proteins present on the surface of donor-derived microvesicles as compared to recipient organ-derived microvesicles, followed by the isolation of donor-derived microvesicles by the detection of an identified specific MHC protein. For example, and not by way of limitation, donor organ-derived microvesicles can be isolated through the use of beads, e.g., magnetic beads, that are conjugated to antibodies that are specific for a protein present on the surface of donor organ-specific microvesicles.

In certain non-limiting embodiments, high exclusion limit agarose-based gel chromatography can be utilized to isolate microvesicles from a recipient's plasma (Taylor et al., 2005). For example, and not by way of limitation, to isolate the total vesicle fraction, the plasma sample can be fractionated using a 2.5×30 cm Sepharose 2B column, run isocratically with PBS, and the elution can be monitored by absorbance at 280 nm. The fractions comprising microvesicles can be concentrated to 2 ml using an Amicon ultrafiltration stirred cell with a 500K Dalton cut-off membrane and can used for the affinity separation of organ-specific microvesicles subpopulations. Since microvesicles within the circulation are generated from multiple cell types, affinity-based approaches can be used to specifically purify subsets of microvesicles (Taylor et al., 2005). For immunosorbent isolation of transplant organ derived microvesicle populations, recipient plasma microvesicles can be selectively incubated with antibodies specific for the donor's MHC profile coupled with magnetic microbeads. After incubation for 2 hours at 4° C., the magnetic bead complexes can be placed in the separator's magnetic field and the unbound microvesicles can be removed with the supernatant. The bound donor-derived microvesicle subsets can be recovered and diluted in IgG elution buffer (Pierce Chemical Co), centrifuged and resuspended in PBS. Donor microvesicle concentration and size distribution can be determined using the NanoSight NS300. Additional methods to isolate microvesicles include, but are not limited to, ultracentrifugation and sucrose gradient-based ultracentrifugation. In certain embodiments, the microvesicle isolation kit, ExoQuickTM, and/or the Exo-FlowTM system from System Bioscience, Inc. can be used.

In certain embodiments, the microvesicles isolated from a biological sample can be enriched for those originating from a specific cell type, for example, but not limited to, lung, pancreas, stomach, intestine, bladder, kidney, ovary, testis, skin, colorectal, breast, prostate, brain, esophagus, liver, placenta or fetus cells. In addition, the microvesicles often carry surface molecules such as antigens from their donor cells, surface molecules, e.g., proteins, can be used to identify, isolate and/or enrich for microvesicles from a specific cell type (Al-Nedawi et al., 2008; Taylor and Gercel-Taylor, 2008). In certain embodiments, the microvesicles can be isolated from a biological sample of a subject and enriched for those originating from the cell of the organ that was transplanted into the subject.

In certain embodiments, microvesicles can be isolated based on the proteins residing on the surface of the microvesicles, which are specific to the cell type from which it originated. For example, and not by way of limitation, the surface antigen can be epithelial-cell-adhesion-molecule (EpCAM), which is specific to microvesicles from cells of lung, colorectal, breast, prostate, head and neck, and hepatic origin, but not of hematological cell origin can be used to isolate microvesicles (Balzar et al., 1999; Went et al., 2004). In certain embodiments, the surface antigen can be CD24, which is a glycoprotein specific to urine microvesicles, and can be used to isolate and/or purify microvesicles from the kidney (Keller et al., 2007). In certain embodiments, the surface antigen can be the renal glomerular marker, Podocalyxin-1, which can be used to isolate and/or purify microvesicles from the kidney. In certain embodiments, the surface antigen can be the renal epithelial marker, Aquaporin 2, which can be used to isolate and/or purify microvesicles from the kidney.

The isolation of microvesicles from specific cell types and/or donor organs can be accomplished, for example, by using antibodies, aptamers, aptamer analogs or molecularly imprinted polymers specific for a desired surface antigen. In certain embodiments, the surface antigen is specific for a cell type of a specific organ. One non-limiting example of a method of microvesicle separation based on cell surface antigen is provided in U.S. Pat. No. 7,198,923. As described in, e.g., U.S. Pat. Nos. 5,840,867 and 5,582,981, WO 2003/050290 and a publication by Johnson et al., 2008, aptamers and their analogs specifically bind surface molecules and can be used as a separation tool for retrieving cell type-specific microvesicles. Molecularly imprinted polymers also specifically recognize surface molecules as described in, e.g., U.S. Pat. Nos. 6,525,154, 7,332,553 and 7,384,589 and Bossi et al., 2007 and are a tool for retrieving and isolating cell type-specific microvesicles. In certain embodiments, microvesicles can be isolated based on the MHC complex residing on the surface of the microvesicles.

### Protein Detection Techniques

In certain embodiments, the methods of the present disclosure can include the detection of one or more markers specific to the donor and/or the donor organ to confirm the proper isolation and/or purification of donor organ-derived exosomes. In certain embodiments, the marker for donor organ-derived exosomes can be a protein present on the surface and/or within the donor organ-specific isolated microvesicles, e.g., exosomes. Proteins can be isolated from a microvesicle using any number of methods, which are well-known in the art, the particular isolation procedure chosen being appropriate for the particular biological sample. In certain embodiments, the protein can be detected on the surface of the microvesicle.

Methods for the detection of proteins are well known to those skilled in the art and include but are not limited to mass spectrometry techniques, 1-D or 2-D gel-based analysis systems, chromatography, enzyme linked immunosorbent assays (ELISAs), radioimmunoassays (RIA), enzyme immunoassays (EIA), Western Blotting, immunoprecipitation and immunohistochemistry. These methods use antibodies, or antibody equivalents, to detect protein. Antibody arrays or protein chips can also be employed, see for example U.S. Patent Application Nos: 2003/0013208A1; 2002/0155493A1, 2003/0017515 and U.S. Pat. Nos. 6,329,209 and 6,365,418, herein incorporated by reference in their entirety.

ELISA and RIA procedures can be conducted such that a protein standard is labeled (with a radioisotope such as 1251 or 35S, or an assayable enzyme, such as horseradish peroxidase or alkaline phosphatase), and, together with the unlabeled sample of microvesicles, brought into contact with the corresponding antibody, whereon a second antibody is used to bind the first, and radioactivity or the immobilized enzyme assayed (competitive assay). Alternatively, the protein present on and/or within the microvesicles is allowed to react with the corresponding immobilized antibody, radioisotope or enzyme-labeled anti-marker antibody is allowed to react with the system, and radioactivity or the enzyme assayed (ELISA-sandwich assay). Other conventional methods can also be employed as suitable.

The above techniques can be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods can also be employed as suitable.

In certain embodiments, the detection of a protein marker from a donor organ-derived microvesicle sample includes the steps of: contacting the microvesicle sample with an antibody or variant (e.g., fragment) thereof which selectively binds the protein marker, and detecting whether the antibody or variant thereof is bound to the sample. The method can further include contacting the sample with a second antibody, e.g., a labeled antibody. The method can further include one or more steps of washing, e.g., to remove one or more reagents.

It can be desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene can provide a suitable support.

Enzymes employable for labeling are not particularly limited but can be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase can be assayed by measuring the concentration of hydrogen peroxide formed after reaction of the enzyme-labeled antibody with the substrate under controlled conditions well-known in the art.

Other techniques can be used to detect a protein marker according to a practitioner's preference based upon the present disclosure. One such technique is Western blotting (Towbin et al., Proc. Nat. Acad. Sci. 76:4350 (1979)), wherein a suitably treated sample is run on an SDS-PAGE gel before being transferred to a solid support, such as a nitrocellulose filter. Antibodies (unlabeled) are then brought into contact with the support and assayed by a secondary immunological reagent, such as labeled protein A or anti-immunoglobulin (suitable labels including 125I, horseradish peroxidase and alkaline phosphatase). Chromatographic detection can also be used.

Other machine or autoimaging systems can also be used to measure immunostaining results for the marker. As used herein, “quantitative” immunohistochemistry refers to an automated method of scanning and scoring samples that have undergone immunohistochemistry, to identify and quantitate the presence of a specified marker, such as an antigen or other protein. The score given to the sample is a numerical representation of the intensity of the immunohistochemical staining of the sample and represents the amount of target marker present in the sample. As used herein, Optical Density (OD) is a numerical score that represents intensity of staining. As used herein, semi-quantitative immunohistochemistry refers to scoring of immunohistochemical results by human eye, where a trained operator ranks results numerically (e.g., as 1, 2 or 3).

Various automated sample processing, scanning and analysis systems suitable for use with immunohistochemistry are available in the art. Such systems can include automated staining (see, e.g., the Benchmark system, Ventana Medical Systems, Inc.) and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.).

Another method that can be used for detecting protein markers is Western blotting. Immunodetection can be performed with antibody to a protein marker using the enhanced chemiluminescence system (e.g., from PerkinElmer Life Sciences, Boston, Mass.). The membrane can then be stripped and re-blotted with a control antibody, e.g., anti-actin (A-2066) polyclonal antibody from Sigma (St. Louis, Mo.).

Antibodies against protein markers can also be used for imaging purposes, for example, to detect the presence of a donor organ-derived microvesicles in a sample of microvesicles obtained from a recipient's blood. Suitable labels include radioisotopes, iodine (1251, 1211), carbon (14C), sulphur (35S), tritium (3H), indium (112 In), and technetium (99mTc), fluorescent labels, such as fluorescein and rhodamine and biotin. Immunoenzymatic interactions can be visualized using different enzymes such as peroxidase, alkaline phosphatase, or different chromogens such as DAB, AEC or Fast Red.

For in vivo imaging purposes, antibodies are not detectable, as such, from outside the body, and so must be labeled, or otherwise modified, to permit detection. Labels for this purpose can be any that do not substantially interfere with the antibody binding, but which allow external detection. Suitable labels can include those that can be detected by X-radiography, NMR or MRI. For X-radiographic techniques, suitable labels include any radioisotope that emits detectable radiation but that is not overtly harmful to the subject, such as barium or caesium, for example. Suitable labels for NMR and MRI generally include those with a detectable characteristic spin, such as deuterium, which can be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99 m.

The labeled antibody or antibody fragment will then preferentially accumulate at the location of the sample which contains a protein marker. The labeled antibody or variant thereof, e.g., antibody fragment, can then be detected using known techniques. Antibodies for use in the present disclosure include any antibody, whether natural or synthetic, full length or a fragment thereof, monoclonal or polyclonal, that binds sufficiently strongly and specifically to the marker to be detected. An antibody can have a Kd of at most about 10-6M, 10-7M, 10-8M, 10-9M, 10-10M, 10-11M, 10-12M. The phrase “specifically binds” refers to binding of, for example, an antibody to an epitope or antigen or antigenic determinant in such a manner that binding can be displaced or competed with a second preparation of identical or similar epitope, antigen or antigenic determinant.

Antibodies and derivatives thereof that can be used encompasses polyclonal or monoclonal antibodies, chimeric, human, humanized, primatized (CDR-grafted), veneered or single-chain antibodies, phase produced antibodies (e.g., from phage display libraries), as well as functional binding fragments, of antibodies. For example, antibody fragments capable of binding to a marker, or portions thereof, including, but not limited to Fv, Fab, Fab′ and F (ab′) 2 fragments can be used. Such fragments can be produced by enzymatic cleavage or by recombinant techniques. For example, papain or pepsin cleavage can generate Fab or F (ab′) 2 fragments, respectively. Other proteases with the requisite substrate specificity can also be used to generate Fab or F (ab′) 2 fragments. Antibodies can also be produced in a variety of truncated forms using antibody genes in which one or more stop codons have been introduced upstream of the natural stop site. For example, a chimeric gene encoding a F (ab′) 2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10:1455-1460 (1992), regarding primatized antibody, and Ladner et al., U.S. Pat. No. 4,946,778 and Bird, R. E. et al., Science, 242:423-426 (1988)) regarding single-chain antibodies.

In certain embodiments, agents that specifically bind to a polypeptide other than antibodies are used, such as peptides. Peptides that specifically bind can be identified by any means known in the art, e.g., peptide phage display libraries. Generally, an agent that is capable of detecting a marker polypeptide, such that the presence of a marker is detected and/or quantitated, can be used. As defined herein, an “agent” refers to a substance that is capable of identifying or detecting a protein marker in a sample (e.g., identifies or detects the mRNA of a marker, the DNA of a marker, the protein of a marker). In certain embodiments, the agent is a labeled or labelable antibody which specifically binds to a marker polypeptide.

In addition, a protein marker can be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See, for example, U.S. Patent Application Nos: 2003/0199001, 2003/0134304, 2003/0077616, which are herein incorporated by reference.

Mass spectrometry methods are well known in the art and have been used to detect biomolecules, such as proteins (see, e.g., Li et al. (2000) Tibtech 18:151-160; Rowley et al. (2000) Methods 20:383-397; and Kuster and Mann (1998) Curr. Opin. Structural Biol. 8:393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In other In certain embodiments, a gas phase ion spectrophotometer can be used. embodiments, laser-desorption/ionization mass spectrometry is used to analyze the sample. Modem laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait).

For additional information regarding mass spectrometers, see, e.g., Principles of Instrumental Analysis, 3rd edition. Skoog, Saunders College Publishing, Philadelphia, 1985; and Kirk-Othmer Encyclopedia of Chemical Technology, 4th ed. Vol. 15 (John Wiley & Sons, New York 1995), pp. 1071-1094.

Detection of the presence of a marker or other substances can involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of a particular marker. Software programs such as the Biomarker Wizard program (Ciphergen Biosystems, Inc., Fremont, Calif.) can be used to aid in analyzing mass spectra. The mass spectrometers and their techniques are well known to those of skill in the art.

Any person skilled in the art understands, any of the components of a mass spectrometer (e.g., desorption source, mass analyzer, detect, etc.) and varied sample preparations can be combined with other suitable components or preparations described herein, or to those known in the art. For example, in some embodiments a control sample can contain heavy atoms (e.g., 13C) thereby permitting the test sample to be mixed with the known control sample in the same mass spectrometry run.

In certain embodiments, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

### RNA Detection Techniques

In certain embodiments, the methods of the present disclosure can include the detection of one or more markers specific to the donor and/or the donor organ to confirm the proper isolation and/or purification of donor organ-derived exosomes. In certain embodiments, the marker is a nucleic acid, including DNA and/or RNA, contained within the donor organ-specific isolated microvesicles, e.g., exosomes. Nucleic acid molecules can be isolated from a microvesicle using any number of methods, which are well-known in the art, the particular isolation procedure chosen being appropriate for the particular biological sample. In certain instances, with some techniques, it can also be possible to analyze the nucleic acid without extraction from the microvesicle.

In certain embodiments, the detection of nucleic acids present in the microvesicles can be quantitative and/or qualitative. Any method for qualitatively or quantitatively detecting a nucleic acid marker can be used. Detection of RNA transcripts can be achieved, for example, by Northern blotting, wherein a preparation of RNA is run on a denaturing agarose gel, and transferred to a suitable support, such as activated cellulose, nitrocellulose or glass or nylon membranes. Radiolabeled cDNA or RNA is then hybridized to the preparation, washed and analyzed by autoradiography.

Detection of RNA transcripts can further be accomplished using amplification methods. For example, it is within the scope of the present disclosure to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4:80-84 (1994). In certain embodiments, quantitative real-time polymerase chain reaction (qRT-PCR) can be used to detect RNA.

Other known amplification methods which can be utilized herein include, but are not limited to, the so-called “NASBA” or “3SR” technique described in PNAS USA 87:1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42:9-13 (1996) and European Patent Application No. 684315; and target mediated amplification, as described by PCT Publication WO9322461.

In situ hybridization visualization can also be employed. Another method for detecting mRNAs in a microvesicle sample is to detect mRNA levels of a marker by fluorescent in situ hybridization (FISH). FISH is a technique that can directly identify a specific sequence of DNA or RNA in a cell, microvesicle sample or biological sample and therefore enables to visual determination of the marker presence and/or expression in tissue samples. Fluorescence in situ hybridization is a direct in situ technique that is relatively rapid and sensitive. FISH test also can be automated. Immunohistochemistry can be combined with a FISH method when the expression level of the marker is difficult to determine by immunohistochemistry alone.

Alternatively, RNA can be detected on a DNA array, chip or a microarray. Oligonucleotides corresponding to the marker(s) are immobilized on a chip which is then hybridized with labeled nucleic acids of a test sample obtained from a subject. Positive hybridization signal is obtained with the sample containing marker transcripts. Methods of preparing DNA arrays and their use are well known in the art. (See, for example, U.S. Pat. Nos. 6,618,6796; 6,379,897; 6,664,377; 6,451,536; 548,257; US20030157485 and Schena et al. 1995 Science 20:467-470; Gerhold et al. 1999 Trends in Biochem. Sci. 24, 168-173; and Lennon et al. 2000 Drug discovery Today 5:59-65, which are herein incorporated by reference in their entirety). Serial Analysis of Gene Expression (SAGE) can also be performed (See for example U.S. patent application No. 20030215858).

To detect RNA molecules, for example, mRNA can be extracted from the microvesicle sample to be tested, reverse transcribed and fluorescent-labeled cDNA probes are generated. The microarrays capable of hybridizing to a marker, cDNA can then be probed with the labeled cDNA probes, the slides scanned, and fluorescence intensity measured. This intensity correlates with the hybridization intensity and expression levels.

Types of probes for detection of RNA include cDNA, riboprobes, synthetic oligonucleotides and genomic probes. The type of probe used will generally be dictated by the particular situation, such as riboprobes for in situ hybridization, and cDNA for Northern blotting, for example. In certain embodiments, the probe is directed to nucleotide regions unique to the particular marker RNA. The probes can be as short as is required to differentially recognize the particular marker RNA transcripts, and can be as short as, for example, 15 bases; however, probes of at least 17 bases, e.g., 18 bases or better 20 bases can be used. In certain embodiments, the primers and probes hybridize specifically under stringent conditions to a nucleic acid fragment having the nucleotide sequence corresponding to the target gene. As herein used, the term “stringent conditions” means hybridization will occur only if there is at least 95% and at least 97% identity between the sequences.

The form of labeling of the probes can be any that is appropriate, such as the use of radioisotopes, for example, 32P and 35S. Labeling with radioisotopes can be achieved, whether the probe is synthesized chemically or biologically, by the use of suitably labeled bases.

### Kits

In certain non-limiting embodiments, the present disclosure provides for a kit for assessing the conditional state of a transplanted organ in a subject that comprises a means for isolating, purifying and/or detecting one or more donor organ-derived microvesicles.

Types of kits include, but are not limited to, packaged probe and primer sets (e.g., TaqMan probe/primer sets), arrays/microarrays, donor organ and/or donor marker-specific antibodies and antibody-conjugated beads, which further contain one or more probes, primers or other detection reagents for detecting one or more donor organ-derived microvesicles, disclosed herein.

In certain non-limiting embodiments, a kit can comprise a pair of oligonucleotide primers suitable for polymerase chain reaction (PCR) or nucleic acid sequencing, for detecting one or markers of the donor organ-derived microvesicles. A pair of primers can comprise nucleotide sequences complementary to a marker and be of sufficient length to selectively hybridize with said marker. Alternatively, the complementary nucleotides can selectively hybridize to a specific region in close enough proximity 5′ and/or 3′ to the marker position to perform PCR and/or sequencing. Multiple marker-specific primers can be included in the kit to simultaneously assay large number of markers. The kit can also comprise one or more polymerases, reverse transcriptase and nucleotide bases, wherein the nucleotide bases can be further detectably labeled.

In certain non-limiting embodiments, a primer can be at least about 10 nucleotides or at least about 15 nucleotides or at least about 20 nucleotides in length and/or up to about 200 nucleotides or up to about 150 nucleotides or up to about 100 nucleotides or up to about 75 nucleotides or up to about 50 nucleotides in length.

In certain non-limiting embodiments, the oligonucleotide primers can be immobilized on a solid surface or support, for example, on a nucleic acid microarray, wherein the position of each oligonucleotide primer bound to the solid surface or support is known and identifiable.

In certain non-limiting embodiments, a kit can comprise at least one nucleic acid probe, suitable for in situ hybridization or fluorescent in situ hybridization, for detecting the marker and/or the donor organ-derived microvesicles. Such kits will generally comprise one or more oligonucleotide probes that have specificity for various markers.

In certain non-limiting embodiments, a kit can comprise at least one antibody for immunodetection of the marker and/or the donor organ-derived microvesicles and/or for the isolation of the donor organ-derived microvesicles. Antibodies, both polyclonal and monoclonal, specific for a marker, can be prepared using conventional immunization techniques, as will be generally known to those of skill in the art. The immunodetection reagents of the kit can include detectable labels that are associated with, or linked to, the given antibody or antigen itself. Such detectable labels include, for example, chemiluminescent or fluorescent molecules (rhodamine, fluorescein, green fluorescent protein, luciferase, Cy3, Cy5, or ROX), radiolabels (3H, 35S, 32P, 14C, 131I) or enzymes (alkaline phosphatase, horseradish peroxidase).

In certain non-limiting embodiments, the antibody can be provided bound to a solid support, such as a column matrix, an array, or well of a microtiter plate. Alternatively, the support can be provided as a separate element of the kit.

In certain non-limiting embodiments, a kit can comprise one or more primers, probes, microarrays, or antibodies suitable for detecting one or more markers.

In certain non-limiting embodiments, where the measurement means in the kit employs an array, the set of markers set forth above can constitute at least 10 percent or at least 20 percent or at least 30 percent or at least 40 percent or at least 50 percent or at least 60 percent or at least 70 percent or at least 80 percent of the species of markers represented on the microarray.

In certain non-limiting embodiments, a marker detection kit can comprise one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction to detect a marker. A kit can also include additional components or reagents necessary for the detection of a marker, such as secondary antibodies for use in immunohistochemistry. A kit can further include one or more other markers or reagents for evaluating other prognostic factors, e.g., stage of rejection.

In certain non-limiting embodiments, a marker detection kit can comprise one or more reagents and/or tools for isolating donor organ-specific microvesicles from a biological sample. A kit can also include reagents necessary for isolating the protein and/or nucleic acids from the isolated microvesicles.

### Reports, Programmed Computers, and Systems

In certain embodiments, the monitoring of the conditional state of a transplanted organ in a subject based on the isolation, purification and/or detection of donor organ-derived microvesicles, can be referred to herein as a “report”. A tangible report can optionally be generated as part of a testing process (which can be interchangeably referred to herein as “reporting,” or as “providing” a report, “producing” a report, or “generating” a report).

Examples of tangible reports can include, but are not limited to, reports in paper (such as computer-generated printouts of test results) or equivalent formats and reports stored on computer readable medium (such as a CD, USB flash drive or other removable storage device, computer hard drive, or computer network server, etc.). Reports, particularly those stored on computer readable medium, can be part of a database, which can optionally be accessible via the internet (such as a database of patient records or genetic information stored on a computer network server, which can be a “secure database” that has security features that limit access to the report, such as to allow only the patient and the patient's medical practitioners to view the report while preventing other unauthorized individuals from viewing the report, for example). In addition to, or as an alternative to, generating a tangible report, reports can also be displayed on a computer screen (or the display of another electronic device or instrument).

A report can include, for example, an individual's medical history, or can just include size, presence, absence or levels of one or more markers (for example, a report on computer readable medium such as a network server can include hyperlink(s) to one or more journal publications or websites that describe the medical/biological implications). Thus, for example, the report can include information of medical/biological significance as well as optionally also including information regarding the detection of organ donor-derived microvesicles, or the report can just include information regarding the detection of organ donor-derived microvesicles without other medical/biological significance.

A report can further be “transmitted” or “communicated” (these terms can be used herein interchangeably), such as to the individual who was tested, a medical practitioner (e.g., a doctor, nurse, clinical laboratory practitioner, genetic counselor, etc.), a healthcare organization, a clinical laboratory, and/or any other party or requester intended to view or possess the report. The act of “transmitting” or “communicating” a report can be by any means known in the art, based on the format of the report. Furthermore, “transmitting” or “communicating” a report can include delivering a report (“pushing”) and/or retrieving (“pulling”) a report. For example, reports can be transmitted/communicated by various means, including being physically transferred between parties (such as for reports in paper format) such as by being physically delivered from one party to another, or by being transmitted electronically or in signal form (e.g., via e-mail or over the internet, by facsimile, and/or by any wired or wireless communication methods known in the art) such as by being retrieved from a database stored on a computer network server, etc.

In certain exemplary embodiments, the disclosed subject matter provides computers (or other apparatus/devices such as biomedical devices or laboratory instrumentation) programmed to carry out the methods described herein. In certain embodiments, the system can be controlled by the individual and/or their medical practitioner in that the individual and/or their medical practitioner requests the test, receives the test results back, and (optionally) acts on the test results to reduce the individual's disease risk, such as by implementing a disease management system.

The following examples are offered to more fully illustrate the disclosure but are not to be construed as limiting the scope thereof.

### EXAMPLE 1: Detection of Donor Organ Specific Microvesicles in Recipient's Bloodstream

There is a significant microvesicle contribution into the blood from immune cells, platelets, and endothelial cells, which results in a large noise-to-signal ratio when a general microvesicle profiling approach is performed. Similar problem exists with studies analyzing circulating free protein and nucleic acid markers. In addition, free protein and nucleic acid markers are unstable in the circulation, thus requiring a high steady-state for detection, and minor changes over time (essential for monitoring) are difficult to quantify. However, microvesicles and their RNA cargo are extremely stable in the circulation. Therefore, transplant organ-specific microvesicles were isolated from the recipient's bloodstream to define states of rejection and/or injury of a transplanted organ.

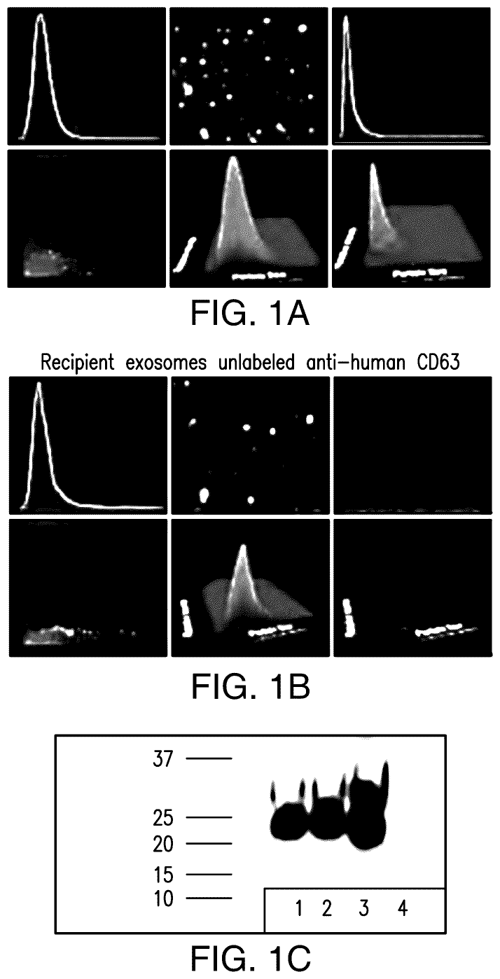

Donor exosomes released from the transplanted organ were isolated in a xenogeneic islet transplant model, where human islets were transplanted into a nude mouse (n=8). Normoglycemic recipients were sacrificed at various time points (range 31 to 198 days), and the donor human islet mass was stained for insulin for histologic confirmation. Exosomes from recipient plasma was analyzed on a NanoSight NS300 with nanoparticle tracking analysis (NTA) upon fluorescent quantum dot labeling with anti-human specific CD63 (exosome marker) antibody. In all 8 recipient animals, anti-human CD63 signal was observed (absent in negative controls) (p=0.006) (FIG. 1A, B). Western blot analysis confirmed detection of human CD63 protein (FIG. 1C).

Similar findings were confirmed using anti-HLA-C specific antibody. This concept was also tested in the allogeneic setting of tolerance (BALB/c islets into diabetic B6 mouse, n=3), with recipients maintaining normoglycemia >300 days. Recipient plasma exosomes showed donor exosome signal (anti-H2-Kd BALB/c specific antibody-quantum dot) signal by NanoSight, which was confirmed by Western blot analysis (absent in negative controls).

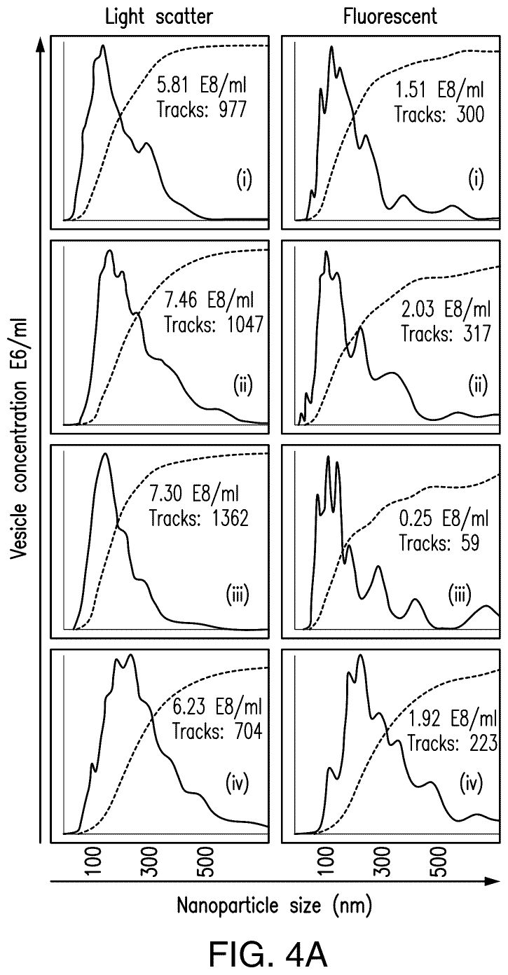

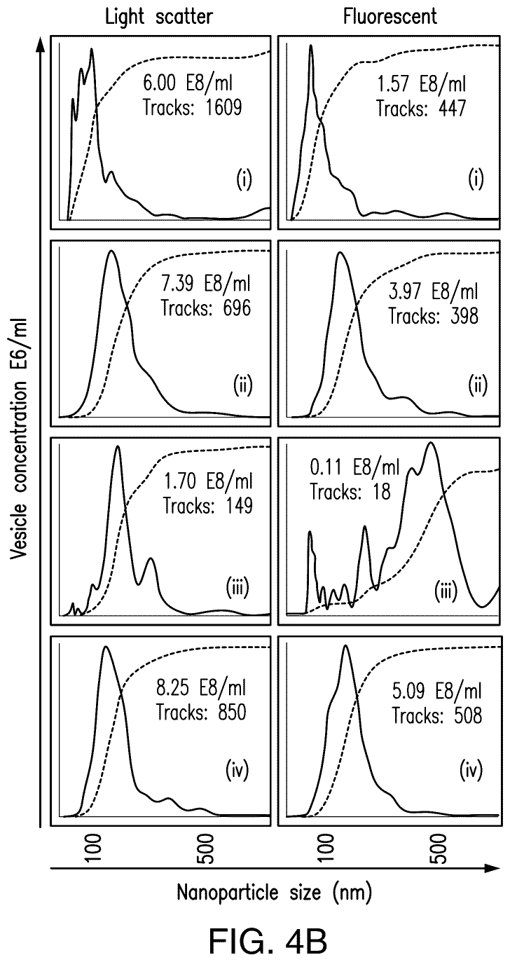

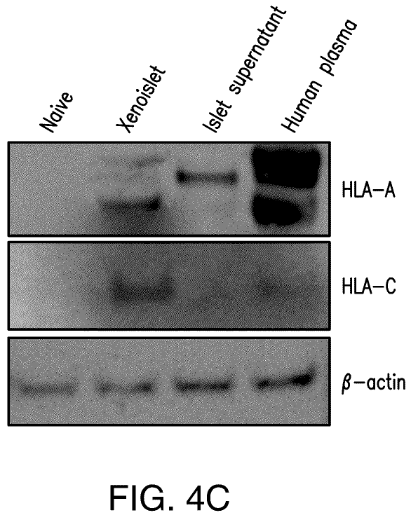

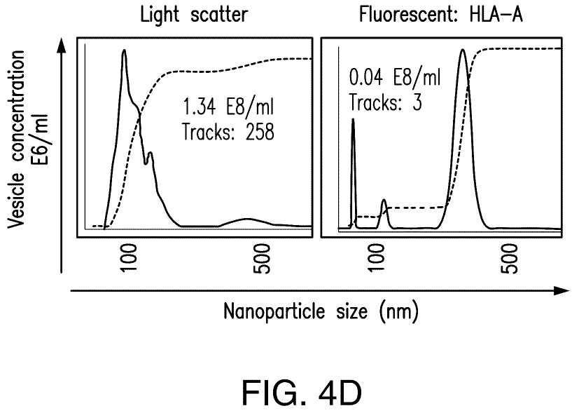

In further experiments, exosomes were isolated from recipient mouse plasma and analyzed for donor islet specific exosomes signal on the NanoSight NS300 nanoparticle detector using anti-human MHC specific antibody-quantum dot. At all tested post-transplant time points, HLA specific exosome signal was detected on NanoSight fluorescence (range 14 to 198 days, n=20) (FIG. 4A, B). This signal was absent in naive mouse (n=5) and in allogeneic mouse islet transplant (n=5) plasma exosome samples (p<0.0001). Western blot analysis showed HLA presence in xenoislet samples (FIG. 4C). To validate transplant exosome signal specificity, islet graftectomy was performed in xenoislet animals and this led to loss of the HLA-A signal (n=6, p<0.001) (FIG. 4D).

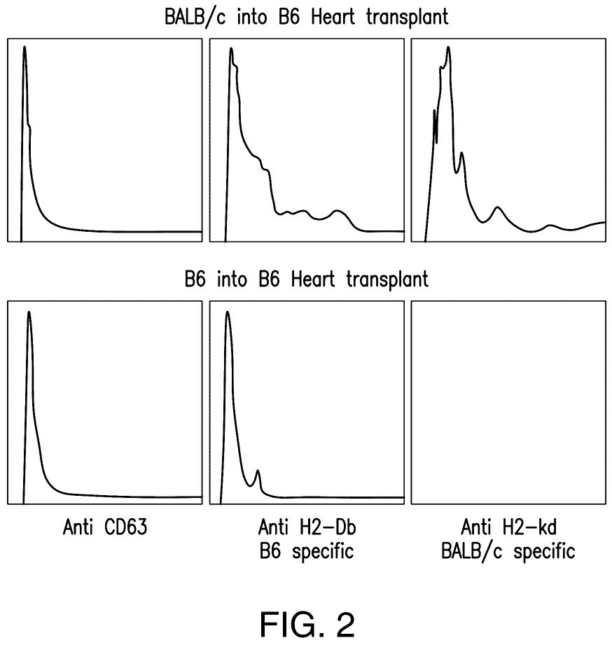

To determine if this concept is applicable with other organ transplants, allogeneic full mismatch heterotopic heart transplants were performed in the mouse, where a BALB/c heart was transplanted into B6 recipient (n=10). In this acute rejection model, recipient animals were sacrificed as early as 4 hours post-transplant to 11 days. Plasma exosome pool was isolated from recipient blood using Sepharose gel filtration chromatography and was assessed at different post-transplant time points for donor heart-specific exosome signal utilizing NanoSight nanoparticle tracking analysis technology with quantum dot labeled anti-donor MHC specific antibodies. At all time points, donor heart-specific exosome signal (anti-H2-Kd BALB/c specific quantum dot) was observed by NanoSight analysis, which was confirmed by Western blot (p=0.003). Western blot was also performed for confirmation. Syngeneic B6 transplants served as negative control (n=3), and the signal was absent in the control animals (FIG. 2).

These studies in mouse models established that transplanted organ/tissue releases a detectable and stable donor tissue specific exosome pool into recipient blood that can be serially tracked over long term follow-up. Donor specific exosomes were detectable in the human clinical setting as well.

### EXAMPLE 2: Characterization of Donor-Derived Microvesicles Isolated from Recipient Patient's Blood and Urine Samples in the Clinical Setting of Human Living Donor Renal Transplantation

To test if transplant tissue specific exosome platform can be translated to other transplant tissues and bodily fluids, donor kidney specific exosomes in recipient patient plasma and urine in the setting of human living donor renal transplantation were isolated.

For analysis of microvesicles in the plasma of a donor kidney recipient, 0.5-2 ml of plasma was obtained through venipuncture and stored at −80° C. For analysis of microvesicles in the urine of a donor kidney recipient, urine samples (40 ml) were collected in sterile cups, treated with 1x protease inhibitor cocktail (Sigma-Aldrich, Co., St. Louis, MO) and frozen at −80° C. until analysis.

Exosome isolation from human plasma samples was performed utilizing 500 ul to 1 ml plasma obtained after centrifugation of the blood sample at 500 g for 10 minutes. The plasma sample was directly added to a Sepharose 2B column and the eluent was collected in 1 ml fractions. The exosome fraction was pooled after monitoring absorbance at 280 nm. The pooled fraction was ultracentrifuged at 110,000 g for 2 hours at 4° C., and the pelleted exosome fraction was resuspended in PBS for downstream analysis. Urinary microvesicle isolation was performed as described elsewhere with slight modification (Rood et al. (2010), Pisitkun et al. (2004)). Briefly, urinary cell debris was removed from 20 ml starting material by centrifugation at 17,000 g for 15 minutes. The supernatant was then ultracentrifuged at 200,000 g for 120 minutes at 4° C. The pellet was resuspended in PBS and loaded onto a Sepharose 2B size exclusion column, and the eluted fractions representing exosomes were pooled. The pooled fractions were concentrated on an Amicon filter (Merck Millipore Ltd., Ireland) with 100 kDa cut-off membrane. The isolated microvesicle pool were then analyzed on the NanoSight NS300.

The isolated microvesicle pool was utilized for affinity separation of donor kidney specific microvesicle subset from the recipient plasma. Similar affinity separation of donor microvesicle subset was performed with the urine samples. HLA cross-matching of all living donor renal transplants provided a panel of anti-donor specific HLA antibodies to utilize for affinity isolation of donor microvesicle pool. Class I (HLA-A, B, or C) mismatch that gave the strongest signal on HLA cross-matching (serotyping antibody binds to donor cell but not recipient cell) was focused on. First, anti-donor HLA antibody on total microvesicle pools from samples using the light and fluorescent (anti-donor HLA antibody-quantum dot) scatter modes on the NanoSight NS300 was tested. This confirmed which antibody can be utilized for affinity based magnetic bead isolation of donor microvesicle pool. The microvesicle subset was then analyzed on NanoSight NS300 NTA on the light scatter mode to quantitate the donor specific microvesicle signal, and on the fluorescent mode (anti-donor HLA antibody-quantum dot) to confirm enrichment of the donor microvesicle population. The unbound fraction from affinity isolation, which represents the recipient microvesicle pool, was also analyzed to confirm the absence of the donor HLA signal on the fluorescent mode.

Microvesicles in urine and plasma were isolated strictly based on the HLA mismatch and the donor specific microvesicles subset was analyzed for organ specific proteins, e.g., renal epithelial cell protein, Aquaporin 2. In addition, anti-donor HLA antibody was utilized to confirm enrichment of the donor specific microvesicle subset, and anti-recipient HLA specific antibody confirmed the absence of the recipient microvesicle in the affinity isolated donor microvesicle subset. Unconjugated HLA allele specific antibodies (mouse anti-HLA-A2,-HLA-B27,-HLA-B13,-HLA-B8) were purchased from One Lambda (CA, USA), for donor HLA class I donor type specific exosome isolation and analysis from recipient plasma and urine.

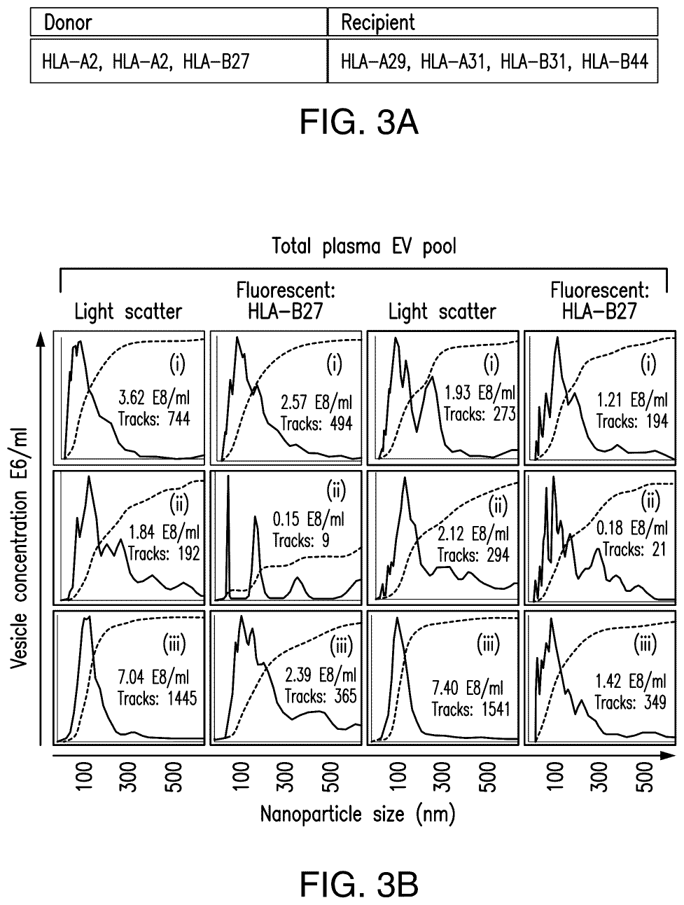

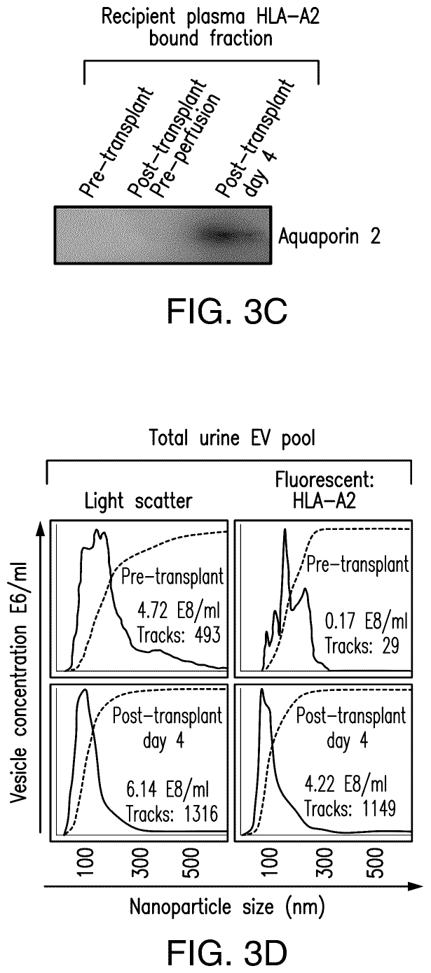

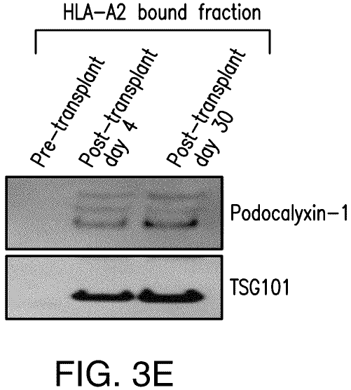

As shown in FIG. 3A, the donor was HLA-A2, HLA-B27 positive and the recipient was HLA-A29, HLA-31, HLA-B31 and HLA-B44 positive. Therefore, anti-donor specific antibodies, HLA-A2 and HLA-B27, were used for detection and purification of donor specific exosomes from recipient plasma. Donor specific exosomes were purified through the use of antibody-conjugated magnetic beads. In brief, a MHC specific antibody was covalently conjugated to N-hydroxysuccinamide magnetic beads (Pierce) per manufacturer's protocol. 50 to 100 ug protein equivalent of exosomes were incubated with antibody beads overnight at 4° C. The bead bound and unbound exosomes fractions were separated per manufacturer's protocol. Exosomes bound to beads were eluted using tris glycine and utilized for downstream analysis.

As shown in FIG. 3B, HLA-A2 and HLA-B27 positive donor kidney specific exosomes were present in recipient patient plasma and in the donor plasma sample as analyzed by NanoSight. Purified exosomes were analyzed on the NanoSight NS300 (405 nm laser diode) on the light scatter mode for exosome quantification and scatter distribution according to manufacturer's protocols (Malvern instruments Inc., MA, USA). Before each experimental run, the machine was calibrated for nanoparticle size and quantity using standardized nanoparticle and dilutions provided by the manufacturer. Surface marker detection on exosomes was performed using the fluorescence mode on the NanoSight NS300. Secondary antibodies conjugated to quantum dots with emission at 605 nm were utilized for fluorescence detection of primary antibodies binding against specific surface proteins (HLA-A, B, C, B27, A2, B13; FXYD2; CD3, CD4, CD8, CD14, CD56, CD19, mouse MHC I) as described previously (Gardiner et al. (2013), Dragovic et al. (2011)). Each experimental run was performed in duplicates, and an appropriate IgG isotype control fluorescence was performed to assess background.

To determine if the exosomes were derived from the proper organ, tissue-specific proteins present on or within the exosomes were analyzed by Western Blot. Western Blot analysis was performed as follows: exosomes and cell lysate total proteins were isolated by pelleting the exosomes and lysing the pellet in 1X RIPA buffer with 1X concentration of protease inhibitor cocktail (Sigma-Aldrich Co., MO). The isolated proteins were separated on polyacrylamide gels and transferred on polyvinylidene difluoride membrane (Life Technologies, NY, USA). The membrane was blocked, incubated with the desired antibody at a concentration per the manufacturer's protocol. Horseradish peroxidase coupled secondary antibody (Santa Cruz Biotechnologies Inc.) was added per manufacturer's protocol and detected through chemiluminescence using Image quant LAS 400 Phospho-Imager (GE Health, USA). Expression of the renal epithelial cell protein, Aquaporin 2, on the donor kidney specific exosome was observed by Western Blot by day 4 post-transplant (FIG. 3C). HLA-A2 bound plasma exosomes fractions from recipient pre-transplant and post-kidney implantation but pre-perfusion of the donor organ was negative for aquaporin 2 (FIG. 3C). These findings were also validated in recipient urine exosomes (FIG. 3D, E). As shown in FIG. 3D, a HLA-A2 signal was absent in the pre-transplant sample but the majority of exosomes from the post-transplant day 4 sample were positive for HLA-A2. By Western Blot, HLA-A2 bound urinary exosomes fractions from postoperative day 4 and day 30 showed expression of renal glomerular marker, podocalyxin-1, but the exosomes from the pre-transplant sample did not (FIG. 3E).

### EXAMPLE 3: Donor tissue specific exosome profiling enables noninvasive monitoring of acute rejection in mouse allogeneic heart transplantation model

### SUMMARY

In heart transplantation there is a critical need for accurate, noninvasive biomarkers to monitor for immunologic rejection, as current clinical standards are based on routine cardiac allograft biopsy. The present study showed that in animal of cardiac allotransplantation, donor heart-specific exosome profiling from recipient plasma can serve as a noninvasive biomarker for diagnosis of early rejection.

Objective. In heart transplantation, there is a need for development of biomarkers to noninvasively monitor the cardiac allograft for immunologic rejection/injury. Exosomes are tissue specific nanovesicles released into circulation by many cell types. Their profiles are dynamic, reflecting conditional changes imposed on their tissue counterparts. Transplanted heart releases donor-specific exosomes into recipient circulation that are conditionally altered during immunologic rejection. This novel concept was investigated in a rodent heterotopic heart transplantation model.

Materials and Methods. Full major histocompatibility (MHC) mismatch [BALB/c (H2-Kd) into C57BL/6 (H2-Kb)] heterotopic heart transplantation was performed in 2 study arms: Rejection (n=64) and Maintenance (n=28) groups. In Rejection arm, immunocompetent recipients fully rejected the donor heart, whereas in Maintenance arm, immunodeficient recipients (C57BL/6 PrkdcSCID) accepted the allograft. Recipient plasma exosomes were isolated, and donor heart-specific exosome signal was characterized on the nanoparticle detector for time specific profile changes using anti-H2-Kd antibody quantum dot.

Results. In Maintenance arm, allografts were viable throughout follow-up of 30 days, with histology confirming absence of rejection/injury. Time course analysis (days 1, 2, 3, 4, 5, 7, 9, 11, 15, 30) showed that total plasma exosome concentration (p=0.157) and donor heart exosome signal (p=0.538) was similar between time points. In Rejection arm, allografts were universally rejected (median day 11). Total plasma exosome quantity and size distribution were similar between follow-up time points (p=0.278). Donor heart exosome signal peaked on day 1, but significantly decreased by day 2 (p=2×10-4) and day 3 (p=3.3×10-6), when histology showed grade OR rejection. Receiver operating characteristic curve for a binary separation of the 2 study arms (Maintenance versus Rejection) demonstrated that donor heart exosome signal threshold <0.3146 was 91.4% sensitive and 95.8% specific for diagnosis of early acute rejection.

Conclusion. Transplant heart exosome profiling enabled noninvasive monitoring of early acute rejection with high accuracy. Application of this concept to the clinical setting could serve as a novel biomarker platform for allograft monitoring in transplantation diagnostics.

### INTRODUCTION

Even with major medical advances, immunologic rejection and immunosuppressive regimen-related complications account for the majority of morbidity and mortality in transplant patients. Recent report by the International Society for Heart and Lung transplantation (ISHLT) shows that the incidence of rejection events in the first-year post-transplant is 25% (Lung 2016). The current gold standard of allograft surveillance comprises empiric surveillance and histology-guided endomyocardial biopsy (EMB), which is associated with procedural complications, and can consume time and resources from patient and health system standpoints (Wong 2008; Sandhu 1989). The frequency of surveillance biopsies varies among centers with routine biopsies typically being performed weekly within the first month post-transplant and increasing intervals thereafter. ISHLT reports that a heart transplant patient undergoes 17 EMBs on average during the first two years post-transplantation. At another institute, heart transplant recipient undergoes 20 surveillance EMBs in the first two years, and that is if there are no rejection episodes.

Exosomes are bilayer membrane-bound nanoparticles (30 to 200 nm) arising from endosomal compartments called multivesicular bodies. They are secreted by many tissue types into bodily fluids, including blood and urine (Vallabhajosyula 2017a; Vallabhajosyula 2017b; Thery 2002). Along with surface marker profiles that are identical to their tissue counterparts, exosomes carry stable proteomic and RNA signatures that are condition-specific. Exosomes are being extensively studied for their diagnostic potential, but similar to other quantitative assays based on circulating free proteins and nucleic acids, whole plasma exosome analysis is also associated with a high noise to signal ratio, as many tissue types contribute to the total plasma exosome pool4. It has been suggested that characterization of tissue-specific exosomes from bodily fluids would improve diagnostic accuracy by reducing noise to signal ratio and therefore serve as a better biomarker. In the context of transplantation, transplant tissue-specific exosome profiling from recipient circulation can serve as an accurate biomarker platform. To characterize transplant tissue exosomes, two concepts were adopted: 1) transplanted tissues release distinct donor major histocompatibility complex (MHC) specific exosomes into recipient circulation, and; 2) iatrogenic donor-recipient MHC mismatch introduced from allotransplantation allows for donor-specific exosome profiling from recipient blood. In the context of heart transplantation, donor heart-specific exosome profiling would enable noninvasive monitoring of immunologic rejection. This was investigated in a heterotopic heart transplantation model.

### Materials and Methods

### Mice

All experiments were conducted in accordance with approved protocols through the University of Pennsylvania Institutional Animal Care and Use Committee (IACUC), and in accordance with the NIH Guide for the Care and Use of Laboratory Animals. C57BL/6 (MHC H2-Kb) immunocompetent wild type and C57BL/6 immunodeficient (PrkdcSCID) mice (MHC H2-Kb) served as recipients. BALB/c (MHC H2-Kd) mice served as heart donors. All animals were purchased from Jackson laboratories (Maine, USA).

Study design

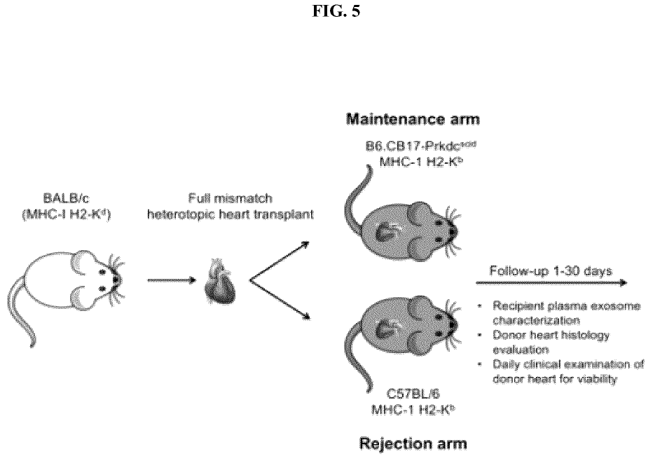

The 2 study groups were Rejection and Maintenance arms. In Rejection arm, full MHC mismatch (BALB/c into C57BL/6) heart transplants were performed, resulting in acute rejection with allograft asystole by day 9 to 12 (median day 11). Recipients were sacrificed for plasma exosome analysis and allograft histology on days 1, 2, 3, 4, 5, 7, 9, 11, 15, and 30. Along with naive pretransplant recipients, at least 5 transplants were performed for each time point (n=64 total). In Maintenance arm, full MHC mismatch heart transplant was performed into C57BL/6 immunodeficient recipient, and since these animals were lack T and B cells, they accepted the allograft long term. Recipients were sacrificed on days pretransplant, 1, 2, 4, 7, 11, and 30 (4 transplants per time point; n=28 total), and recipient plasma exosome and donor heart histological analysis were performed. Schematic of the study design is shown in FIG. 5.

Heterotopic heart transplantation and post-transplant monitoring

Animals were anesthetized with ketamine and xylazine, and donor was heparinized with 200 units of heparin after which heart was explanted. Recipient's abdominal aorta and inferior vena cava was exposed, and the donor heart pulmonary artery to vena cava and donor aorta to recipient aorta anastomoses were performed to complete the allograft implantation. Allograft function was assessed daily via percutaneous palpation and heart rate monitoring until the animal was sacrificed or until asystole. Recipients were sacrificed for plasma and donor heart harvest at mentioned time points. Plasma sample from each animal was analyzed independently.

Plasma exosome isolation

300 to 500 ul of whole blood was centrifuged at 500g to obtain plasma that was passed via size exclusion chromatography column to obtain eluent fractions containing exosomes (Vallabhajosyula 2017a). The pooled fractions were filtered through a 100 kDa cut-off membrane and ultracentrifuged at 120,000 g for 2 hours at 4° C. Pellet containing exosomes was resuspended in phosphate buffered saline (1xPBS) for downstream analysis.

Nanoparticle detector analysis

Exosomes were analyzed on the NanoSight NS300 nanoparticle detector (Malvern Instruments Inc., Massachusetts) for quantitation and size distribution of total plasma exosomes and donor heart specific exosomes. Total plasma exosomes were analyzed on the light scatter mode. For donor heart specific exosome characterization, subpopulation of recipient plasma exosomes with surface expression of donor BALB/c MHC (H2-Kd) was profiled using anti-H2-Kd antibody conjugated quantum dot (Biolegend, California) on the nanoparticle detector fluorescence mode. Mouse IgG antibody quantum dot (Santa Cruz, California) was used as isotype control. Each sample was run in duplicates and each experimental run was duplicated independently. In each displayed panel, the nanoparticle size distribution curve is represented by particle size (nanometers) on x axis and nanoparticle concentration (x106/ml) on y axis. Curve in blue represents the total plasma exosome pool distribution, and the red curve represents the subpopulation of donor tissue specific exosomes.

Donor heart exosome signal was quantified as follows:

| H2-Kd fluorescence- | Pretransplant H2-Kd fluorescence- | IgG isotype fluorescence |
|---|---|---|
| H2-Kd light scatter | Pretransplant light scatter | IgG isotype light scatter |

Western blot

Equal quantities of exosome protein and tissue lysates (10 ug) were run on polyacrylamide gels, transferred on nitrocellulose membrane (Life Technologies, New York). Exosomal fractions and tissue lysate were run on 2 separate blots as indicted by a dashed line in FIG. 2A. The blots were blocked and incubated with the desired primary antibody at concentration per manufacturer's protocol. Horseradish peroxidase coupled secondary antibodies (Cell signaling Technology, Massachusetts) were added and detected through chemiluminescence using ImageQuant LAS 400 phosphoimager.

### Histology

Donor heart tissue was cut with cryostat, fixed with 4% paraformaldehyde and blocked (0.05% Triton X-100) before staining with hematoxylin and eosin. For time course characterization of rejection, T cell infiltration was assessed by immunohistochemistry using anti-CD3 antibody. Analysis was performed using Zeiss epifluorescence microscope. Histological evaluation and ISHLT grading of acute rejection in donor heart was performed by the clinical heart transplant pathologist.

Statistical analysis

First, data were checked for normality. Statistical significance for parametric data was assessed by independent sample t test (2-tailed) for continuous variables with 2 groups (donor heart-specific exosome signal day 1 versus day 2, donor heart-specific exosome signal day 2 versus day 3, donor heart-specific exosome signal day 1 versus day 3, all in Rejection arm) and one-way analysis of variance (ANOVA) for continuous variable with more than 2 groups (normality and homoscedasticity assumed, for donor heart-specific exosome signal in Maintenance arm, donor heart-specific exosome signal in Rejection arm from day 3 to 30 and total exosome concentrations from pretransplant time-point to day 30 for both Maintenance and Rejection arm). Additionally, all comparative statistical tests for total exosome concentration and donor heart-specific signal characterization (independent sample t-test, one-way ANOVA) were performed using Monte Carlo Permutation data resampling. This was done to further statistically validate the significance of the findings with respect to total exosome concentration and donor heart specific exosome analysis.

Therefore, p values are reported as “p” and “permute p” for results of Monte Carlo Permutation data resampling analysis and considered significant if <0.05. For receiver operating characteristic (ROC) curve, the true-positive rate (sensitivity) was plotted against the false-positive rate (specificity) to illustrate performance of a binary classifying system (Maintenance versus Rejection arms). A threshold was determined using the Youden index, and likelihood ratio, sensitivity and specificity were calculated. ROC curves were compared using the method of Delong et al (DeLong 1988). General statistics were assessed using StataMP version 14.2 (StataCorp LP, Texas), and scatter plots and NanoSight panels were constructed using Prism version 7.0 (GraphPad, California). All reported tests were 2-tailed and alpha level was set to 0.05.

### Results

Donor BALB/c MHC is specifically expressed on BALB/c tissues and their exosomes

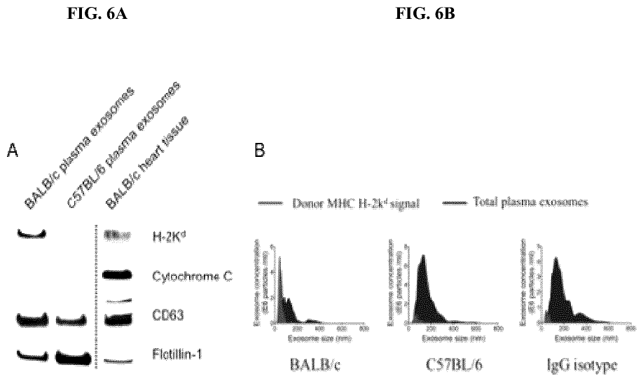

It was confirmed that plasma exosomes isolated from naive donor BALB/c mice express H2-Kd MHC class I antigens on their surface and that the signal is undetectable in both naive C57BL/6 wild type and C57BL/6 immunodeficient (PrkdcSCID) recipient naïve mouse plasma exosomes. First, to validate exosomes were successfully isolated using the described methodologies, Western blot analysis of plasma exosome protein content was performed. In both BALB/c and C57BL/6 exosomes, expression of canonical exosome markers flotillin-1 and CD63 was noted, without expression of cytosolic marker cytochrome c (FIG. 6A). In addition, BALB/c exosomes specifically expressed H2-Kd MHC compared to C57BL/6 exosomes. To demonstrate MHC-specific surface expression, nanoparticle detector analysis was performed. This showed that MHC class I antigens were expressed on exosome surface and were specific to the MHC of their tissue counterparts (FIG. 6B).

Heterotopic heart transplantation

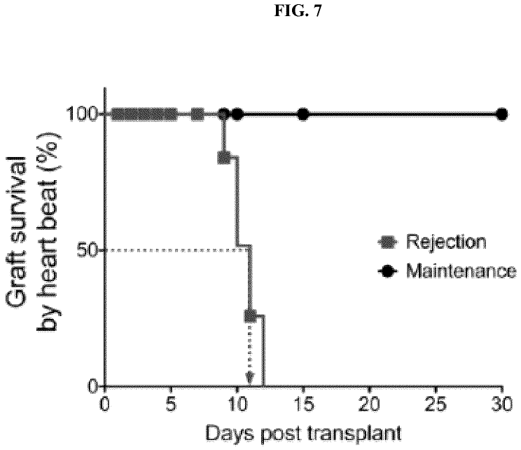

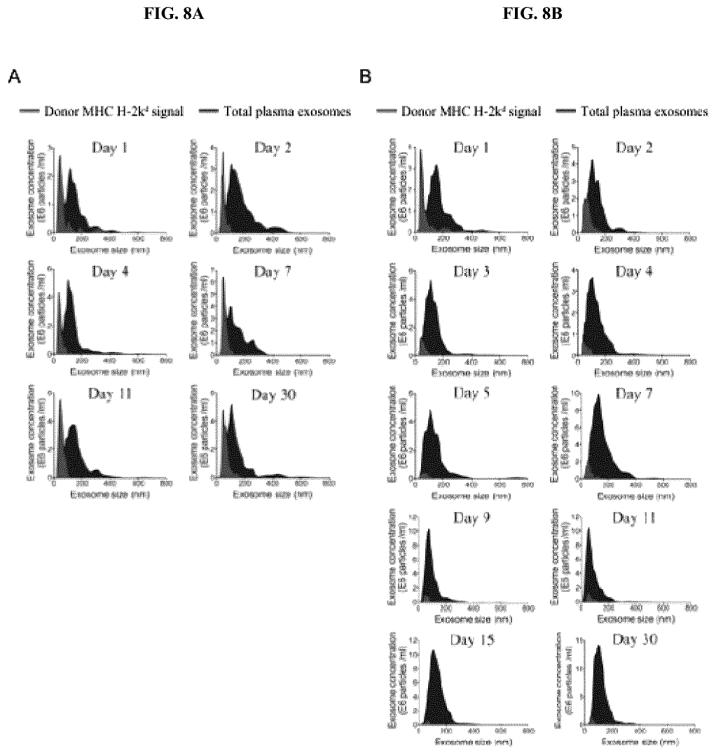

Given above results, full MHC-mismatched heart transplants were performed in both study arms. In Maintenance arm, recipients showed normal allograft function through follow-up (FIG. 7). In Rejection arm, deterioration in function (by abdominal palpation) was noted with asystole occurring between days 9 to 12 (median 11 days) (FIG. 7). On nanoparticle detector analysis, total plasma exosome distribution and quantity was similar at all measured time points in the Maintenance arm (FIG. 8A); and donor BALB/c heart exosome signal distribution was also similar throughout follow-up (FIG. 8A). On the Rejection arm, total plasma exosomes were similar at all time points, but the donor heart exosome signal decreased significantly by day 2, which further dropped by day 3 and remained low at levels equivalent to naive recipient controls (FIG. 8B).

Donor heart exosome signal heralds early acute rejection

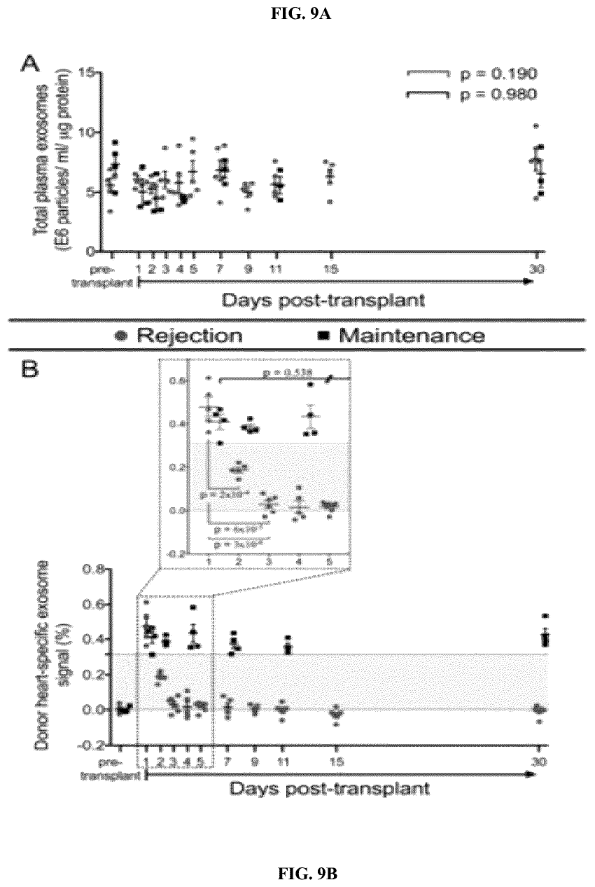

In light of the changes in donor heart exosome signal with immunologic rejection, the time sensitivity and accuracy of the transplant tissue exosome platform in this model were assessed. First, it was checked whether total plasma exosomes show immunologic rejection-specific or time-specific changes in their profiles. In both study arms, at all tested time points including pre-transplant samples, total plasma exosome quantities were similar (p=0.278 for Rejection arm, p=0.157 for Maintenance arm by omnibus testing) (FIG. 9A). This demonstrated that total plasma exosome quantitation was neither sensitive nor specific for noninvasive monitoring of the cardiac allograft conditional status, and immunologic rejection does not alter total plasma exosome quantity or size distribution.

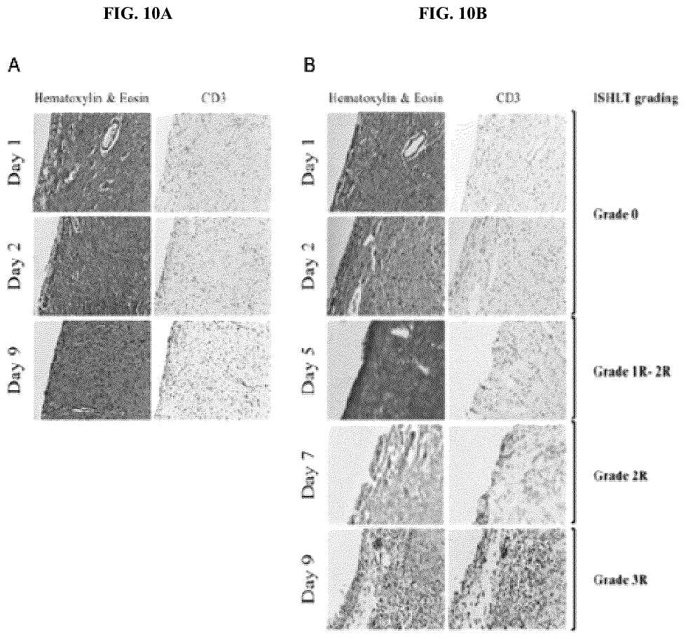

Next, the accuracy of donor heart exosome profiling was assessed. To validate specificity, BALB/c exosome signal in naive pretransplant C57BL/6 wild type and C57BL/6 immunodeficient animals was tested, and absence of BALB/c exosome signal was noted (FIG. 9B pretransplant values). However, donor exosome signal was detectable through all post-transplant time points in the Maintenance arm (p=0.538 by ANOVA), validating signal specificity (FIG. 9B). The donor heart exosome signal remained stable over follow-up, suggesting that stable allograft function without rejection leads to stable donor heart exosome signal in the recipient plasma. Given this, it was assessed whether immunologic rejection would lead to changes in the transplant heart exosome signal. The kinetics of plasma donor heart exosomes during allograft rejection was compared to time-matched samples in the Maintenance arm (FIG. 10B). On day 1, donor heart exosome signals were similar between the two groups (p=0.280), however significant decrease in the signal was noted by 2 in the Rejection arm only (p=2x10-4). Donor exosome signal further decreased on day 3 (p=3x10-6 compared to day 1; p=6x10-5 compared to day 2) but was unchanged in the Maintenance arm. The donor exosome signal remained low and unchanged after day 3 in the Rejection arm (p=0.217 for day 3 to day 30 by ANOVA), similar to the pretransplant levels (FIG. 9B). In order to statistically further validate the diagnostic potential of the donor heart exosome platform compared to total plasma exosome analysis, permutation testing of the total exosome values and donor heart exosome signal values in the Rejection and Maintenance arms was performed. Results of permutation testing showed no differences in total exosome quantities over follow-up within each group and between the two study arms. But it validated that there was significant difference in donor heart exosome signals between days 1 to 2, 1 to 3, and 2 to 3 (Table 1). Taken together, this demonstrated that donor heart exosome characterization enables noninvasive detection of early acute rejection with high sensitivity and specificity in this model.

TABLE 1
Independent sample T-test/one-way ANOVA omnibus testing
versus Monte Carlo Permutation data resampling

| | P value | P value (Monte |
|---|---|---|
| | (ANOVA/ | Carlo |
| Variables | T-test) | Permutation)_ |
| Total exosome concentration | 0.157 | 0.320 |
| Maintenance arm | | |
| (pretransplant to day 30) | | |
| Total exosome concentration | 0.278 | 0.290 |
| Rejection arm | | |
| (pretransplant to day 30) | | |
| Total exosome concentration | 0.254 | 0.241 |
| Maintenance versus | (by T-test) | by logistic |
| Rejection arm | | regression |
| Donor heart-specific exosome | 0.280 | 0.552 |
| signal day 1 Maintenance | | |
| versus day 1 Rejection | | |
| Donor heart-specific exosome | 0.538 | 0.336 |
| signal Maintenance arm | | |
| (day 1 to 30) | | |
| Donor heart-specific exosome | 0.0002 | 0.0076 |
| signal day 1 versus day 2 | (2 × 10−4) | (7.6 × 10−3) |
| Donor heart-specific exosome | 0.00006 | 0.004 |
| signal day 2 versus day 3 | (6 × 10−5) | (4 × 10−3) |
| Donor heart-specific exosome | 0.000003 | 0.0045 |
| signal day 1 versus day 3 | (3 × 10−6) | (4.5 × 10−3) |
| Donor heart-specific exosome | 0.217 | 0.265 |
| signal Rejection arm | | |
| (day 3 to 30) | | |

As the current standard for monitoring rejection in cardiac transplantation is based on histological grading of ventricular EMB, it was investigated the biomarker potential and time sensitivity of the transplant tissue exosome platform in comparison to histology-based grading criteria of acute rejection. Allograft myocardium was analyzed by H&E staining and by immunohistochemistry for T cell infiltration using anti-CD3 antibody. On the Maintenance arm, no signs of allograft injury or T cell infiltration was noted at any of the time points (FIG. 10A). On the Rejection arm, grade 0 rejection was noted for days 1 and 2 (FIG. 10B), when the donor heart exosome signal had already significantly decreased (FIG. 8B and FIG. 9B). Grade 1R to 2R rejection was seen by day 5 and progressed to grade 3R rejection by day 9. Multifocal T cell infiltration was evident in day 7 to 9 histology specimens. These histological results were in accordance to the transplant heart functional status, which revealed median time of allograft activity of 11 days. Collectively, these results demonstrated that donor heart exosome signal significantly decreases before histological evidence of early myocyte injury and T cell infiltration (grade 1R).

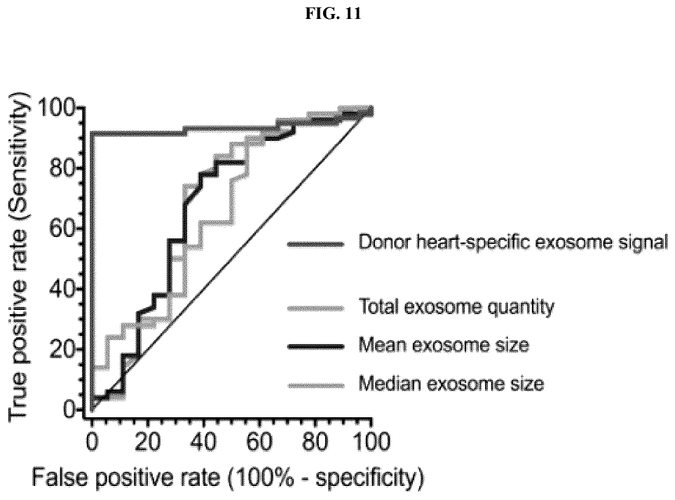

Given these promising results, a receiver operating characteristic (ROC) curve was generated to statistically assess the biomarker potential of transplant heart exosome platform to predict early acute rejection (<grade 1R). ROC curves were generated for a binary outcome using all Rejection arm time points (day 1 to 30) as rejection, and all Maintenance arm time points (day 1 to 30) as no rejection. Area under the curve (AUC) values for total exosome quantity (AUC-0.659+0.080), median exosome size (AUC-0.677+0.085), and mean exosome size (AUC=0.679+0.083) showed poor diagnostic accuracy (FIG. 11). Donor heart exosome profiling showed that a signal threshold cut-off of <0.3146 was 91.4% sensitive and 95.8% specific (AUC-0.934+0.030) with a likelihood ratio of 21.9 for diagnosing acute rejection in this model (FIG. 11). Taken together, this validated the superior biomarker potential of donor heart specific exosome profiling for time sensitive diagnosis of early rejection, compared to whole plasma exosome analysis.

### DISCUSSION

Current clinical gold standard for diagnosis and surveillance of acute and chronic cellular rejection remains histological analysis by EMB. To date no biomarker-based platform has been widely accepted in the clinical setting of heart transplantation. Although there are few biomarker platforms that are being studied, such as peripheral blood mononuclear cell gene expression profiling (Allomap, Pham 2010) and donor derived cell free DNA quantitation (De Vlaminck 2014), these methodologies have not infiltrated clinical practice to replace EMB as the first-line diagnostic. Measurement of circulating protein markers such as CK, CK-MB, and troponin as biomarkers, which are routinely used for diagnosis of myocardial infarction, are of little value for timely, accurate diagnosis of acute cardiac allograft rejection (Dengler 1998). The present investigation relates to an entirely different and novel biomarker platform in heart transplantation, one based on profiling of transplant tissue specific exosomes from recipient circulation. It was demonstrate that: 1) cardiac allograft released a distinct pool of donor MHC specific exosomes into recipient circulation that was maintained at a steady state under conditions of allograft acceptance and immunodeficiency, 2) circulating donor heart exosomes could be tracked and quantified reliably under conditions of allograft maintenance, and, 3) acute immunologic rejection led to time-sensitive and accurate changes in donor heart exosome profiles, thus enabling noninvasive monitoring for condition specific changes causing allograft injury.

Two other biomarker platforms are being extensively studied for noninvasive cardiac allograft monitoring. The Allomap test, based on profiling of expression of 11 genes in peripheral blood mononuclear cells, was studied in a randomized trial for noninferiority to EMB in distinguishing grade 0 (quiescence) from grade ≥2R rejection (moderate to severe rejection) 8. Interestingly, the study excluded patients in the first 6 to 12 months after heart transplantation, when the risk of acute cellular rejection is greatest. The test correctly identified 84% of moderate to severe rejection episodes. Although the study reported noninferiority on their composite primary outcome, the results showed that gene-expression profiling strategy can be associated with up to 68% increase in risk (hazard ratio 1.04, 95% confidence interval 0.67 to 1.68). Furthermore, of the 34 rejection episodes detected in the patients randomized to Allomap testing surveillance, only 6 rejection episodes were detected on the basis of elevated gene expression score. Therefore, the Allomap test has been adopted by some cardiac transplant programs for monitoring in patients beyond the first 6 to 12 months after transplantation, but it has not gained wide acceptance in the clinical realm to replace EMB.

Another platform being studied is quantitation of circulating donor-derived cell free DNA (ddcfDNA) (De Vlaminck 2014; Synder 2011). Unlike the Allomap test, this platform has potential universal application in transplantation diagnostics, as it is based on the concept that acute rejection leads to increased levels of ddcfDNA. Two studies investigating this idea in heart transplant patients showed that ddcfDNA enabled distinction of grade 0 versus moderate to severe rejection on ROC analysis with an AUC=0.83, sensitivity of 58% and specificity of 93% for ddcfDNA threshold of 0.25% (De Vlaminck 2014; Dengler 1998). Whereas the AUC was 0.6 for distinguishing grade 0 versus mild (grade 1R) rejection. Furthermore, the accuracy of the test worsened in patients of older age. Therefore, although ddcfDNA platform holds promise for development of non-invasive diagnostic platform in heart transplantation, these data demonstrate that other novel biomarker platforms should be investigated.

Conceptually, profiling circulating cardiac allograft exosomes encompasses the strengths associated with the above noninvasive diagnostics, and addresses some of the concerns associated with them. Like ddcfDNA, the platform disclosed in the present study also has universal application. Recently, the diagnostic potential of profiling transplant islet tissue specific exosomes as a novel biomarker platform in islet transplantation was reported. The validated extension of this platform in kidney transplantation was also reported, showing successfully quantified donor kidney-specific exosomes from recipient blood and urine (Vallabhajosyula 2017a). Unlike ddcfDNA quantities, which exist at very low levels in the blood and require relatively complex methodologies, donor heart exosome signal levels were easily detectable using simple methodologies in this study. Whereas other studies distinguished grade 0 from moderate to severe rejection, the platform enabled detection of early stages of acute rejection with high accuracy. Lastly, donor heart exosome profiling provides another layer of characterization that enables further improvement of its accuracy and time sensitivity. In addition to quantitative changes, the proteomic and RNA (especially microRNA) cargoes of exosomes are also altered based on the conditional stimulus/stress placed on their tissue counterparts. MicroRNAs are small, non-coding RNAs that bind to the 3′ UTR of target messenger RNAs (mRNAs). Although outside the scope of this study, it had previously demonstrated that acute rejection of transplanted tissue leads to distinct changes in their exosome protein and RNA signatures (Vallabhajosyula 2017a). Future studies analyzing the exosome cargoes of transplanted heart specific exosomes might reveal distinct microRNA and proteomic markers associated with cardiac allograft rejection. If so, this would further improve the accuracy and time sensitivity of the non-invasive platform.

Towards a better understanding of microRNA cargo profiles in the context of solid organ transplantation, a recent study by Dewi et al. (Sukma 2017) found a significant increase of exosomal microRNA miR-142-3p content in the total serum exosome pool of heart transplant recipients under conditions of acute cellular rejection (ACR) versus no rejection. ACR was also associated with differential regulation and enrichment of miR-92a-3p and miR-339-3p (Sukma 2017). Further analyses suggested that miR-142-3p, a hematopoietic tissue-specific microRNA, is released from T cells upon stimulation by means of exosomal transport (miR-142-3p significantly increased in the exosomal fraction but not in supernatant of T cell culture) and could modify expression of its target gene mRNA, RAB11FIP2, in endothelial cells (Sukma 2017). Such a T cell-endothelial cell axis via an exosomal shuttle may contribute to T cell mediated graft rejection. The ability of exosomes to shuttle functional microRNA between cells implicates their functional roles in gene regulation, gene expression, and intercellular communication. Studies characterizing transplant heart exosome RNA cargoes can facilitate understanding of their diagnostic potential, and their functional roles in the immunologic rejection process. Exosomes plays a significant role in donor antigen trafficking and in the formation of the immune synapse (Liu 2016, Campana 2015).

Lastly, significant decrease in the signal occurred early in the rejection process, when histology still showed grade 0 (quiescence) rejection without any myocyte damage. In accordance to these findings, similar changes in donor exosome profiles in a murine xenogeneic islet transplantation model was also noted, where transplant tissue exosome levels decreased before any injury to the transplanted islet mass (Vallabhajosyula 2017a). Since the output measured is circulating transplant exosome levels, mechanistically it is not known whether the signal drop early during rejection is due to decreased production of exosomes by the transplanted tissue, increased consumption of the transplant exosomes by recipient immune cells/other cell types, or a combination of both. If this is a consumptive process mediated by immune cells, then to some extent the data in the Maintenance arm argues against this idea as the recipients in this group were deficient of T cells and B cells, and yet the transplant heart exosome levels reached steady state by day 1 and remained at the same level as day 1 levels in the Rejection arm, without further increasing. One would expect the levels to rise more in an immunodeficient state, rather than reaching steady state so early. A recent report showed that as part of the acute rejection process, donor exosomes are important for presentation of donor antigens to recipient alloreactive T cells in lymphoid organs by the recipient dendritic cells in a phenomenon labeled as “cross-dressing” (Liu 2016; Campana 2015; Burlingham 2017; Markey 2014). This mechanism of alloreactive T cell activation, which can occur early during the rejection process, may mediate suppression of further production of exosomes by the transplanted tissue even before there is targeted injury to the allograft. As exosomes are important mediators of donor tissue-immune cell interactions, future studies investigating the mechanistic aspects of this interface may open avenues for targeted therapeutics using donor specific exosomes to manipulate the recipient immune response. Lastly, in this investigation immunologic rejection-specific changes in transplant heart exosomes was examined. The recipient immune cell specific exosomes profiling can further improve the diagnostic accuracy of tissue-specific exosome platform. During injury to transplanted heart with acute rejection, there is also stimulation of alloreactive T cells, which may lead to increased production of T cell exosomes. Concomitant characterization of T cell-specific exosome profiles would further enhance accuracy of this platform. T cell exosome cargo analysis may also provide mechanistic insights into the physiologic roles of exosomes in the immune synapse.

In summary, the present study validates a novel biomarker platform based on transplant tissue specific exosome profiling that can have clinical translational potential in the field of cardiac transplantation diagnostics. The animal model demonstrated that donor heart exosome profiles predicted early acute rejection with high accuracy. These results suggest the application of the biomarker in the clinical setting.

### EXAMPLE 4: Characterization of Circulating Donor Heart Specific Exosomes in Clinical Heart Transplantation

There is a critical need for novel biomarker development for monitoring rejection in heart transplantation. Exosomes are tissue specific nanovesicles with dynamic profiles that reflect condition-specific changes imposed on their tissue counterparts. In animal transplant models it was demonstrated that donor tissue specific exosome profiles enable noninvasive diagnosis of rejection. The present study investigated the translational potential of this platform in clinical heart transplantation.

Peripheral blood samples were prospectively collected during the perioperative period in 5 patients undergoing heart transplantation (preoperative time point to day 30). Exosomes were isolated using size exclusion chromatography and ultracentrifugation. Transplant heart specific exosomes (THEs) were quantified on nanoparticle detector using anti-donor human leukocyte antigen I (HLA) specific antibody-quantum dot. THEs were enriched using anti-donor HLA I antibody conjugated beads, and their intraexosomal cargo was analyzed for cardiac marker Troponin I. In one patient with day 14 biopsy-proven antibody mediated rejection (AMR), THEs were analyzed for complement protein C4 deposition.

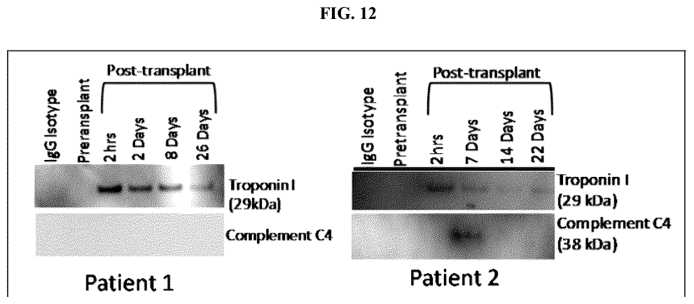

In all 5 patients, on nanoparticle detector THEs were only detected in the post-transplant samples not pretransplant (p<0.001), even at the earliest measured time point of 2 hours post-implantation. Purified THEs showed expression of Troponin I protein and mRNA in post-transplant samples, validating THE enrichment (FIG. 12). In patient with AMR, THEs also showed time specific expression of complement C4 (not observed in other 4 patients) (FIG. 12).

Transplanted heart released THEs into recipient circulation at least within 2 hours after implantation. Donor specific HLA I antibodies enabled quantitation of THE signal, and profiling of THE protein and RNA intraexosomal cargo. THE profiles enable noninvasive characterization of transplant heart injury in the clinical setting.

### EXAMPLE 5. Donor Lung Specific Exosome Profiles for Noninvasive Monitoring of Acute Rejection in a Rat Orthotopic Left Lung Transplant Model

There is a critical need for development of biomarkers to monitor for lung transplant rejection. Circulating donor lung specific exosome profiles as biomarkers for time sensitive diagnosis of acute rejection in a rat orthotopic lung transplant model was investigated.

Left lung from Wistar transgenic rat expressing exosome marker human specific CD63-GFP was transplanted into Lewis recipient across full major histocompatibility complex mismatch (n=12). Recipient blood was collected daily (days 0 to 8) and plasma was processed for exosome isolation by size exclusion chromatography and ultracentrifugation. Donor lung specific exosomes were profiled using anti-human CD63 antibody quantum dot on the nanoparticle detector. Fluorescence microscopy for GFP was performed to understand donor lung exosome trafficking during the acute rejection process.

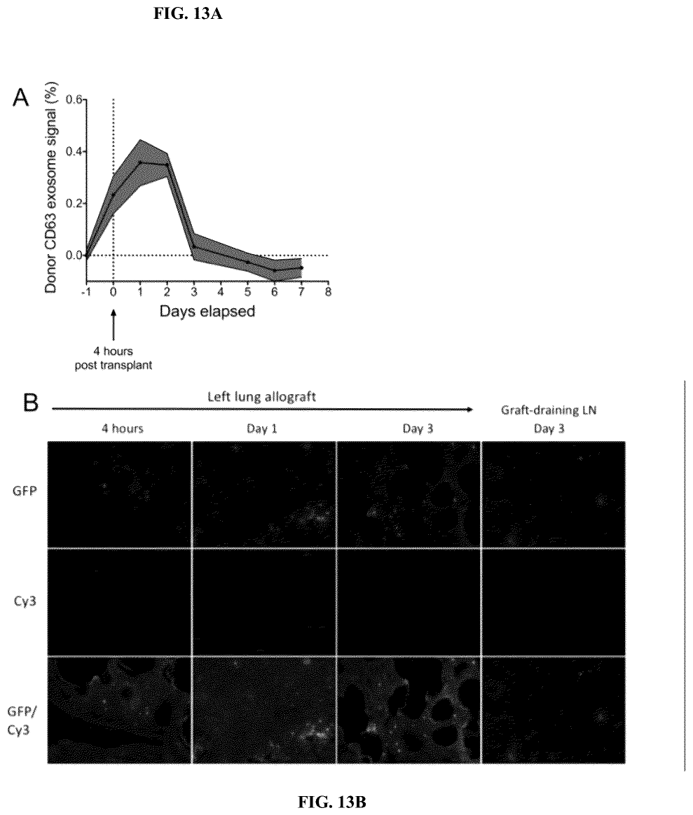

Donor left lung was rejected with loss of perfusion by computer tomography imaging by day 7. Donor lung specific exosomes were detected at all post-transplant time points. Total plasma exosome quantities, mean and median particle size were similar at all time points. Donor lung exosome signal peaked on day 1 and day 2 post-transplant, and then consistently decreased (FIG. 13A). Donor lung exosome signal decreased to near-baseline levels by day 3 and remained low over follow-up. Histology showed that change in donor lung exosome signal preceded onset of early acute rejection (day 4). Confocal microscopy demonstrated human CD63-GFP exosome signal in the left lung and graft-draining lymphoid tissues, suggesting trafficking of the transplant lung exosomes (FIG. 13B).

Transplanted lung released tissue specific exosomes into recipient circulation; their profiles enabled time sensitive diagnosis of acute rejection in this model. Transplant tissue specific exosome can form biomarker platform in lung transplantation.

### REFERENCES

Al-Nedawi, K., B. Meehan, J. Micallef, V. Lhotak, L. May, A. Guha, and J. Rak. 2008. Intercellular transfer of the oncogenic receptor EGFRvIII by microvesicles derived from tumour cells. Nat Cell Biol. 10:619-24.
Aquino-Dias, E. C. et al. Non-invasive diagnosis of acute rejection in kidney transplants with delayed graft function. Kidney Int. 73, 877-884 (2008).
Balzar, M., M. J. Winter, C. J. de Boer, and S. V. Litvinov. 1999. The biology of the 17-1A antigen (Ep-CAM). J Mol Med. 77:699-712.
Bossi, A., F. Bonini, A. P. Turner, and S. A. Piletsky. 2007. Molecularly imprinted polymers for the recognition of proteins: the state of the art. Biosens Bioelectron. 22:1131-7.
Burlingham W J. “Cross-Dressing” Becomes Fashionable Among Transplant Recipients. Am J Transplant. 2017; 17:5-6.
Campana S, De Pasquale C, Carrega P, Ferlazzo G, Bonaccorsi I. Cross-dressing: an alternative mechanism for antigen presentation. Immunol Lett. 2015; 168:349-354.
Cheruvanky, A., H. Thou, T. Pisitkun, J. B. Kopp, M. A. Knepper, P. S. Yuen, and R. A. Star. 2007. Rapid isolation of urinary exosomal biomarkers using a nanomembrane ultrafiltration concentrator. Am J Physiol Renal Physiol. 292: F1657-61.
De Vlaminck I, Valantine H A, Snyder T M, Strehl C, Cohen G, Luikart H, et al.
Circulating cell-free DNA enables noninvasive diagnosis of heart transplant rejection. Sci Transl Med. 2014; 6: 241ra277.
Dengler T J, Zimmermann R, Braun K, Muller-Bardorff M, Zehelein J, Sack F U, et al.
Elevated serum concentrations of cardiac troponin T in acute allograft rejection after human heart transplantation. J Am Coll Cardiol. 1998; 32:405-412.
Delcayre A., Shu H., Le Pecq J. B. Dendritic cell-derived Exosomes in Cancer Immunotherapy: Exploiting Nature's Antigen Delivery Pathway. Expert Rev. Anti-Cancer Therapy 5 (3), in press (2005).
DeLong E R, DeLong D M, Clarke-Pearson D L. Comparing the areas under two or more correlated receiver operating characteristic curves: a nonparametric approach. Biometrics. 1988; 44:837-845.
Dragovic, R. A. et al. Sizing and phenotyping of cellular vesicles using Nanoparticle Tracking Analysis. Nanomedicine. 7 (6), 780-788 (2011).
Escudier B, Dorval T, Chaput N et al. Vaccination of metastatic melanoma patients with autologous dendritic cell (DC) derived exosomes: results of the first phase I clinical trial. J. Transl. Med. 3 (1), 10 (2005).
Gardiner, C., Ferreira, Y. J., Dragovic, R. A., Redman, C. W. and Sargent, I. L. Extracellular vesicle sizing and enumeration by nanoparticle tracking analysis. J Extracell Vesicles. 2, 19671 (2013).
Johnson, S., D. Evans, S. Laurenson, D. Paul, A. G. Davies, P. K. Ferrigno, and C. Walti. 2008. Surface-immobilized peptide aptamers as probe molecules for protein detection. Anal Chem. 80:978-83.
Keller, S., C. Rupp, A. Stoeck, S. Runz, M. Fogel, S. Lugert, H. D. Hager, M. S. Abdel-Bakky, P. Gutwein, and P. Altevogt. 2007. CD24 is a marker of exosomes secreted into urine and amniotic fluid. Kidney Int. 72:1095-102.
Kotsch, K. et al. Enhanced granulysin mRNA expression in urinary sediment in early and delayed acute renal allograft rejection. Transplantation. 77, 1866-1875 (2004).
Li, B. et al. Noninvasive diagnosis of renal-allograft rejection by measurement of messenger RNA for perforin and granzyme B in urine. N Engl J Med. 344, 947-954 (2001).
Liu Q, Rojas-Canales D M, Divito S J, Shufesky W J, Stolz D B, Erdos G, et al. Donor dendritic cell-derived exosomes promote allograft-targeting immune response. J Clin Invest. 2016; 126:2805-2820.
Lund L H, Edwards L B, Dipchand A I, Goldfarb S, Kucheryavaya A Y, Levvey B J, et al. The Registry of the International Society for Heart and Lung Transplantation: Thirty-third Adult Heart Transplantation Report-2016; Focus Theme: Primary Diagnostic Indications for Transplant. J Heart Lung Transplant. 2016; 35:1158-1169.
Markey K A, Koyama M, Gartlan K H, Leveque L, Kuns R D, Lineburg K E, et al. Cross-dressing by donor dendritic cells after allogeneic bone marrow transplantation contributes to formation of the immunological synapse and maximizes responses to indirectly presented antigen. J Immunol. 2014; 192:5426-5433.
Morse M A, Garst J, Osada T, et al. A phase I study of dexosome immunotherapy in patients with advanced non-small cell lung cancer. J. Transl. Med. 3 (1), 9 (2005).
Pisitkun, T., Shen, R-F. & Knepper, M. A. Identification and proteomic profiling of exosomes in human urine. Proc Natl Acad Sci USA. 101 (36), 13368-13373 (2004).
Pham M X, Teuteberg J J, Kfoury A G, Starling R C, Deng M C, Cappola T P, et al. Gene-expression profiling for rejection surveillance after cardiac transplantation. N Engl J Med. 2010; 362:1890-1900.
Raposo, G., H. W. Nijman, W. Stoorvogel, R. Liejendekker, C. V. Harding, C. J. Melief, and H. J. Geuze. 1996. B lymphocytes secrete antigen-presenting vesicles. J Exp Med. 183:1161-72.
Rood, I. M. et al. Comparison of three methods for isolation of urinary microvesicles to identify biomarkers of nephrotic syndrome. Kidney Int. 78 (8), 810-6 (2010).
Sandhu J S, Uretsky B F, Zerbe T R, Goldsmith A S, Reddy P S, Kormos R L, et al. Coronary artery fistula in the heart transplant patient. A potential complication of endomyocardial biopsy. Circulation. 1989; 79:350-356.
Snyder T M, Khush K K, Valantine H A, Quake S R. Universal noninvasive detection of solid organ transplant rejection. Proc Natl Acad Sci USA. 2011; 108:6229-6234.
Sukma Dewi I, Celik S, Karlsson A, Hollander Z, Lam K, McManus J W, et al. Exosomal miR-142-3p is increased during cardiac allograft rejection and augments vascular permeability through down-regulation of endothelial RAB11FIP2 expression. Cardiovasc Res. 2017; 113:440-452.
Suthanthiran, M. et al. Urinary-cell mRNA profile and acute cellular rejection in kidney allografts. N Engl J Med. 369, 20-31 (2013).
Tatapudi, R. R. et al. Noninvasive detection of renal allograft inflammation by measurements of mRNA for IP-10 and CXCR3 in urine. Kidney Int. 65, 2390-2397 (2004).
Taylor, D. D., and C. Gercel-Taylor. 2008. MicroRNA signatures of tumor-derived exosomes as diagnostic biomarkers of ovarian cancer. Gynecol Oncol. 110:13-21.
Taylor, D. D., Gercel-Taylor, C. Tumor-derived exosomes as mediates of T-cell signaling defects. British Journal of Cancer. 2005. 92:305-311.
Taylor, D. D., Zacharias, W., and Gercel-Taylor, C. Exosome isolation for proteomic analyses and RNA profiling. In: Methods in Molecular Biology (RJ Simpson, ed). 2011. 728:235-246.
Théry C, Zitvogel Z, Amigorena S. Exosomes: composition, biogenesis and function. Nat. Rev. Immunol. 2 (8), 569-579 (2002).
Thery C, Zitvogel L, Amigorena S. Exosomes: composition, biogenesis and function. Nat Rev Immunol. 2002; 2:569-579.
Vallabhajosyula P, Korutla L, Habertheuer A, Yu M, Rostami S, Yuan C X, et al. Tissue-specific exosome biomarkers for noninvasively monitoring immunologic rejection of transplanted tissue. J Clin Invest. 2017a.
Vallabhajosyula P, Korutla L, Habertheuer A, Reddy S, Schaufler C, Lasky J, et al. Ex Vivo Lung Perfusion Model to Study Pulmonary Tissue Extracellular Microvesicle Profiles. Ann Thorac Surg. 2017b.
Wong R C, Abrahams Z, Hanna M, Pangrace J, Gonzalez-Stawinski G, Starling R, et al. Tricuspid regurgitation after cardiac transplantation: an old problem revisited. J Heart Lung Transplant. 2008; 27:247-252.
Zitvogel L, Regnault A, Lozier A et al. Eradication of established murine tumors using a novel cell-free vaccine: dendritic cell-derived exosomes. Nat. Med. 4 (5), 594-600 (1998).

Various publications, patents and patent applications are cited herein, the contents of which are hereby incorporated by reference herein in their entireties

What is claimed is:

1. A method comprising:
(a) obtaining a biological sample from a human subject that has received a heart transplant, wherein the sample comprises subject-derived and donor-derived microvesiscles;
(b) producing a fraction of the sample of (a) by
(i) isolating microvesicles from about 30 nm to about 200 nm in size; and
(ii) purifying donor-derived microvesicles by using a donor-specific anti-major histocompatibility complex (MHC) antibody, wherein the fraction is enriched with donor-derived microvesicles;
(c) measuring a number of donor-derived microvesicles in the fraction of (b);
(d) identifying the subject as having an increased risk of transplant rejection when the number of donor-derived microvesicles measured in (c) is below a reference level; and
(e) administering an effective amount of immunosuppressor to the subject.

2. The method of claim 1, wherein the biological sample is selected from the group consisting of a blood sample or a urine sample.

3. The method of claim 2, wherein the biological sample is a blood sample.

4. The method of claim 1, further comprising measuring expression of a cardiac marker in an intraexosomal cargo of the donor-derived microvesicles.

5. The method of claim 4, wherein the cardiac marker is a Troponin I.

6. The method of claim 1, wherein the microvesicles are isolated by high exclusion limit agarose-based gel chromatography.

7. A method of monitoring the state of a heart transplant, the method comprising:
(a) obtaining a first biological sample and a second biological sample from a human subject that has received the transplant, wherein each of the first sample and the second sample comprises subject-derived and donor-derived microvesiscles and wherein the first sample and the second sample are obtained at different times;
(b) producing a fraction of the first sample and of the second sample of (a) by
(i) isolating microvesicles from about 30 nm to about 200 nm in size; and
(ii) purifying donor-derived microvesicles by using a donor-specific anti-major histocompatibility complex (MHC) antibody, wherein the fraction is enriched with donor-derived microvesicles;
(c) measuring a number of donor-derived microvesicles in each fraction of (b);
(d) identifying the subject as having an increased risk of transplant rejection when the number of donor-derived microvesicles measured in the second sample is below the number of donor-derived microvesicles measured in the first sample; and
(e) administering an effective amount of immunosuppressor to the subject.

8. The method of claim 7, wherein each of the first biological sample and the second biological sample is selected from the group consisting of a blood sample or a urine sample.

9. The method of claim 8, wherein each of the first biological sample and the second biological sample is a blood sample.

10. The method of claim 7, further comprising measuring expression of a cardiac marker in an intraexosomal cargo of the donor-derived microvesicles in each of the first biological sample and the second biological sample.

11. The method of claim 10, wherein the cardiac marker is a Troponin I.

12. The method of claim 7, wherein the microvesicles are isolated by high exclusion limit agarose-based gel chromatography.
